(19) United States
(12) Patent Application Publication
(10) Pub. No.: US 2022/0096529 A1
(43) Pub. Date: Mar. 31, 2022

(54) 2-HYDROXYPROPYL-BETA-CYCLODEXTRIN (HPBETACD) FOR USE IN THE TREATMENT OF BREAST CANCER

(71) Applicant: UNIVERSITY OF THE WITWATERSRAND, JOHANNESBURG, JOHANNESBURG (ZA)

(72) Inventors: Mandeep KAUR, Johannesburg (ZA); Sourav Taru SAHA, Johannesburg (ZA)

(73) Assignee: UNIVERSITY OF THE WITWATERSRAND, JOHANNESBURG, JOHANNESBURG (ZA)

(21) Appl. No.: 17/426,780

(22) PCT Filed: Jan. 28, 2020
(86) PCT No.: PCT/IB2020/050651

§ 371 Date: Jul. 29, 2021

(30) Foreign Application Priority Data
Jan. 30, 2019 (ZA) 2019/00623

(51) Int. Cl. A61K 31/724; A61P 35/00

(52) U.S. Cl. CPC A61K 31/724; A61P 35/00

(57) ABSTRACT

A method including the step of administering 2-hydroxypropyl-β-cyclodextrin (HPβCD) for use in the treatment of breast cancer. Particularly, this invention relates to HPβCD for use in the treatment of triple negative breast cancer, wherein the HPβCD is for administration to a patient in need thereof. The invention extends to methods of preparing a pharmaceutical composition comprising HPβCD, and further extends to a method of treating breast cancer, typically triple negative breast cancer, by administration of the composition to a patient in need thereof.

### FIELD OF INVENTION

This invention relates to 2-hydroxypropl-β-cyclodextrin (HPβCD) for use in the treatment of breast cancer. Particularly, this invention relates to HPβCD for use in the treatment of triple negative breast cancer, wherein the HPβCD is for administration to a patient in need thereof. The invention extends to methods of preparing a pharmaceutical composition comprising HPβCD, and further extends to a method of treating breast cancer, typically triple negative breast cancer, by administration of said composition to a patient in need thereof.

### BACKGROUND

Cancer is one of the leading causes of deaths worldwide. According to WHO, the number of new cancer cases worldwide is expected to rise by about 70% over the next two decades. Breast cancer is one of the most commonly occurring malignancies in women around the world, with almost 1.7 million new cases identified in 2012. In 2012, over 8 000 new cases of breast cancer were diagnosed in South Africa, making up 21.79% of the total cancer diagnoses nationally (National Health Laboratory Service, 2012).

Current treatments available for breast cancer include use of selective estrogen receptor modulator (SERM) drugs which include tamoxifen (a gold standard drug) which is known to have several side effects. These side effects include the development of uterine cancer, cataracts, blood clots and heart attacks. Moreover, over a period of time patients develop resistance to drugs. Thus, alternative therapeutic approaches needs to be investigated for successful cancer treatment.

Africa and Asia have the least number of breast cancer survivors when compared to Northern America and Europe. Without being limited to theory, this anomaly is believed to include a genetic component. It has been seen that in African and Asian populations breast cancer patients are not receptive to typical hormone based treatments. African and Asian populations most frequently display so-called triple negative breast cancer when compared to other populations. Triple negative breast cancer means that the three most common types of receptors known to fuel most breast cancer growth, namely, estrogen, progesterone and HER-2/neu gene, are not present in the cancer tumor. As such, commonly used pharmaceutically active ingredients (API) designed to target such receptors are ineffective. Triple negative breast cancer has no known effective treatment protocol, leaving its sufferers with little or no chance of recovery.

This is particularly inimical in African and Asian populations where access to medicine is often hampered, and poverty exacerbates lack of access and treatment. Consequently, there is a dire need to develop effective and cost effect pharmaceutical compositions and/or treatment protocols for breast cancer, specifically triple negative breast cancer.

The invention described herein below strives to ameliorate at least one of the problems described above and/or otherwise known in the prior art.

### SUMMARY

Broadly, and in accordance with a first aspect of this invention there is provided 2-hydroxypropyl-β-cyclodextrin (HPβCD) for use in the treatment of breast cancer in a human or animal body.

Preferably the breast cancer is triple negative breast cancer.

The 2-hydroxypropyl-β-cyclodextrin (HPβCD) may be formulated as a pharmaceutical composition.

The pharmaceutical composition may be for administration via a parenteral and/or non-parenteral route.

Parenteral administration may include, but is not limited to, intravenous, intramuscular, or implantation into the human or animal body.

Non-parenteral administration may include, but is not limited to, oral, rectal, vaginal, sublingual, buccal, and intranasal delivery of the pharmaceutical composition into the human or animal body.

The pharmaceutical composition may include an excipient.

The pharmaceutical composition may include additional active pharmaceutical ingredients.

The pharmaceutical composition may include further cyclodextrins from the family of cyclodextrins.

In accordance with a second aspect of this invention there is provided a pharmaceutical composition comprising 2-hydroxypropyl-β-cyclodextrin (HPβCD) for use in the treatment of breast cancer in a human or animal body.

Preferably the breast cancer is triple negative breast cancer.

The pharmaceutical composition may be formulated for administration via a parenteral and/or non-parenteral route.

Parenteral administration may include, but is not limited to, intravenous, intramuscular, or implantation into the human or animal body.

Non-parenteral administration may include, but is not limited to, oral, rectal, vaginal, sublingual, buccal, and intranasal delivery of the pharmaceutical composition into the human or animal body.

The pharmaceutical composition may include an excipient.

The pharmaceutical composition may include additional active pharmaceutical ingredients.

The pharmaceutical composition may include further cyclodextrins from the family of cyclodextrins.

In accordance with a third aspect of this invention there is provided use of 2-hydroxypropyl-β-cyclodextrin (HPβCD) in the manufacture of a pharmaceutical composition for the treatment of breast cancer in a human or animal body.

Preferably the breast cancer is triple negative breast cancer.

The pharmaceutical composition may be formulated for administration via a parenteral and/or non-parenteral route.

Parenteral administration may include, but is not limited to, intravenous, intramuscular, or implantation into the human or animal body.

Non-parenteral administration nay include, but is not limited to, oral, rectal, vaginal, sublingual, buccal, and intranasal delivery of the pharmaceutical composition into the human or animal body.

The pharmaceutical composition may include an excipient.

The pharmaceutical composition may include additional active pharmaceutical ingredients.

The pharmaceutical composition may include further cyclodextrins from the family of cyclodextrins.

In accordance with a fourth aspect of this invention there is provided a method of treating breast cancer, said method comprising the step of administering 2-hydroxypropyl-β-cyclodextrin (HPβCD) to a human or animal in need thereof.

Preferably the breast cancer is triple negative breast cancer.

The 2-hydroxypropyl-β-cyclodextrin (HPβCD) may be formulated as a pharmaceutical composition.

The pharmaceutical composition may be for administration via a parenteral and/or non-parenteral route.

Parenteral administration may include, but is not limited to, intravenous, subcutaneous, intramuscular, or implantation into the human or animal body.

Non-parenteral administration may include, but is not limited to, oral, rectal, vaginal, sublingual, buccal, and intranasal delivery of the pharmaceutical composition into the human or animal body.

The pharmaceutical composition may include an excipient.

The pharmaceutical composition may include additional active pharmaceutical ingredients.

The pharmaceutical composition may include further cyclodextrins from the family of cyclodextrins.

In accordance with a fifth aspect of this invention there is provided a method of inducing and/or facilitating apoptosis of breast cancer cells, the method including the step of contacting said breast cancer cells with 2-hydroxypropyl-β-cyclodextrin (HPβCD) and/or a pharmaceutical composition comprising 2-hydroxypropyl-β-cyclodextrin (HPβCD) in a human or animal body.

The method wherein the breast cancer cells may be triple negative breast cancer cells.

The method wherein the step of contacting said breast cancer cells with 2-hydroxypropyl-β-cyclodextrin (HPβCD) and/or the pharmaceutical composition comprising 2-hydroxypropyl-f-cyclodextrin (HPβCD) includes the administration of 2-hydroxypropyl-β-cyclodextrin (HPβCD) and/or the pharmaceutical composition comprising 2-hydroxypropyl-β-cyclodextrin (HPβCD) by parenteral and/or non-parenteral means to the human or animal body.

There is further provided for 2-hydroxypropyl-β-cyclodextrin (HPβCD) for use according to the first aspect of this invention above, substantially as herein described, illustrated and/or exemplified with reference to any one of the figures and/or examples hereunder.

There is further provided for a pharmaceutical composition according to the second aspect of this invention above, substantially as herein described, illustrated and/or exemplified with reference to any one of the figures and/or examples hereunder.

There is further provided for use of 2-hydroxypropyl-β-cyclodextrin (HPβCD) in the manufacture of a pharmaceutical composition according to a third aspect of the invention above, substantially as herein described, illustrated and/or exemplified with reference to any one of the figures and/or examples hereunder.

There is further provided for a method of treating breast cancer according to a fourth aspect of this invention above, substantially as herein described, illustrated and/or exemplified with reference to any one of the figures and/or examples hereunder.

There is further provided for a method of treating breast ca inducing and/or facilitating apoptosis of breast cancer cells according to a fifth aspect of this invention above, substantially as herein described, illustrated and/or exemplified with reference to any one of the figures and/or examples hereunder.

### BRIEF DESCRIPTION OF THE DRAWINGS

FIG. 1: Representative graph comparing the percentage growth inhibition of MCF7 cells at selected concentrations of HPβCD at 1 mM, 5 mM, 10 mM, 20 mM and 50 mM. Plumbagin (40 μM) was used as a positive control. Data are mean±standard deviation S.D. (n=3) from raw data, where *p<0.05, **p<0.01 and ***p<0.001 significant difference to untreated control.

FIG. 2: Representative graph comparing the percentage growth inhibition of MDA-MB-231 cells at selected concentrations of HPβCD at 1 mM, 5 mM, 10 mM, 20 mM and 50 mM. Plumbagin was used as a positive control. Data are mean±standard deviation S.D. (n=3) from raw data, where *p<0.05, **p<0.01 and ***p<0.001 significant difference to untreated control.

FIG. 3: Representative graph comparing the percentage growth inhibition of MRC-5 cells at selected concentrations of HPβCD at 1 mM, 5 mM, 10 mM, 20 mM and 50 mM. Plumbagin was used as a positive control. Data are mean±standard deviation S.D. (n=3) from raw data, where *p<0.05, **p<0.01 and ***p<0.001 significant difference to untreated control.

FIG. 4: Representative graph comparing the percentage growth inhibition of HEK-293 cells at selected concentrations of HPβCD at 1 mM, 5 mM, 10 mM, 20 mM and 50 mM. Plumbagin was used as a positive control. Data are mean±standard deviation S.D. (n=3) from raw data, where *p<0.05, **p<0.01 and ***p<0.001 significant difference to untreated control.

FIG. 5: Representative graph comparing the percentage of apoptosis in MCF7 cells at selected concentrations of HPβCD at 1 mM, 5 mM, 10 mM, 20 mM, 50 mM and negative samples. Plumbagin was used as a positive control. Data are mean±standard deviation S.D. (n=3) from raw data, where *p<0.05, **p<0.01 and ***p<0.001 significant difference to untreated control.

FIG. 6: Light microscope images showing the amount of apoptosis in MCF7 cells at selected concentrations of HPβCD at 1 mM, 5 mM, 10 mM, 20 mM, 50 mM and negative samples. Plumbagin was used as a positive control.

FIG. 7: Representative graph comparing the percentage of apoptosis in MDA-MB-231 cells at selected concentrations of HPβCD at 1 mM, 5 mM, 10 mM, 20 mM 0.50 mM and negative samples. Plumbagin was used as a positive control. Data are mean±standard deviation S.D. (n=3) from raw data, where *p<0.05, **p<0.01 and ***p<0.001 significant difference to untreated control.

FIG. 8: Light microscope images showing the amount of apoptosis in MDA-MB-231 cells at selected concentrations of HPβCD at 1 mM, 5 mM, 10 mM, 20 mM, 50 mM and negative samples. Plumbagin was used as a positive control.

FIG. 9: Representative graph comparing the percentage of apoptosis in MRC-5 cells at selected concentrations of HPβCD at 1 mM, 5 mM, 10 mM, 20 mM, 50 mM and negative samples. Plumbagin was used as a positive control. Data are mean 2 standard deviation S.D. (n=3) from raw data, where *p<0.05, **p<0.01 and ***p<0.001 significant difference to untreated control.

FIG. 10: Light microscope images showing the amount of apoptosis in MRC-5 cells at selected concentrations of HPβCD at 1 mM, 5 mM, 10 mM, 20 mM, 50 mM and negative samples. Plumbagin was used as a positive control.

FIG. 11: Representative graph comparing the percentage of apoptosis in HEK-293 cells at selected concentrations of HPβCD at 1 mM, 5 mM, 10 mM, 20 mM, 50 mM and negative samples. Plumbagin was used as a positive control. Data are mean±standard deviation S.D. (n=3) from raw data, where *p<0.05, **p<0.01 and ***p<0.001 significant difference to untreated control.

FIG. 12: Light microscope images showing the amount of apoptosis in HEK-293 cells at selected concentrations of HPβCD at 1 mM, 5 mM, 10 mM, 20 mM, 50 mM and negative samples. Plumbagin was used as a positive control.

FIG. 13 A to C: MOMP plots for MCF7 cells. (A): Loss of mitochondrial membrane potential in untreated cells, (B): Loss of mitochondrial membrane potential in positive sample (10 mM H₂O₂) and (C): Loss of mitochondrial membrane potential at 10 mM HPβCD.

FIG. 14 A to C: MOMP plots for MDA-MB-231 cells. (A): Loss of mitochondrial membrane potential in untreated cells. (B): Loss of mitochondrial membrane potential in positive sample (10 mM H₂O₂) and (C): Loss of mitochondrial membrane potential at 10 mM HPβCD.

FIG. 15 A to C: MOMP plots for MRC-5 cells. (A): Loss of mitochondrial membrane potential in untreated cells, (B): Loss of mitochondrial membrane potential in positive sample (10 mM H₂O₂) and (C): Loss of mitochondrial membrane potential at 10 mM HPβCD.

FIG. 16 A to C: ROS generation in MCF7 cells. (A): ROS generation in untreated cells (7.6%) (B): ROS generation in positive sample (10 mM H₂O₂) (88.5%) and (C): ROS generation at 10 mM HPβCD (17.9%).

FIG. 17 A to C: ROS generation in MDA-MB-231 cells (A): ROS generation in untreated cells (3.7%), (B): ROS generation in positive sample (10 mM H₂O₂) (73%) and (C): ROS generation at 10 mM HPβCD (13.7%).

FIG. 18 A to C: ROS generation in MRC-5 cells. (A): ROS generation in untreated cells (2.8%). (B): ROS generation in positive sample (10 mM H₂O₂) (64.1%) and (C): ROS generation at 10 mM HPβCD (10.8%).

FIG. 19 A to C: Caspase 3/7 profile in MCF cells. (A): Total apoptotic/live cells generation in untreated cells. Total Apoptotic—6.4%. (B): Total apoptotic/live cells generation in positive sample (40 μM Plumbagin), Total Apoptotic—23.55% and (C): Total apoptotic/live cells in treated (10 mM HPβCD) cells, Total Apoptotic—12.5%.

FIG. 20 A to C: Caspase 3/7 profile in MDA-MB-231 cells. (A): Total apoptotic/live cells generation in untreated cells (Total Apoptotic 19.6%) (B): Total apoptotic/live cells generation in positive sample (40 μM Plumbagin) (Total Apoptotic—80.45%) and (C): Total apoptotic/live cells generation in treated (10 mM HPβCD) cells (Total Apoptotic—48.75%).

FIG. 21 A to C: Caspase 3/7 profile in HEK-293 cells. (A): Total apoptotic/live cells generation in untreated cells (Total Live—71.90%), (B): Total apoptotic/live cells generation in positive sample (40 μM Plumbagin)(Total Apoptotic—72.40%) and (C): Total apoptotic/live cells generation in treated (10 mM HPβCD) cells (Total Live—66.75%).

FIG. 22: Representative graph comparing the total, free and esterified cholesterol levels in MCF7 cells at selected concentrations of HPβCD at 1 mM, 5 mM, 10 mM, 20 mM, 50 mM and negative samples. Plumbagin was used as a positive control. Data are mean±standard deviation S.D. (n=3) from raw data, where *p<0.05, **p<0.01 and ***p<0.001 significant difference to untreated control.

FIG. 23: Representative graph comparing the total, free and esterified cholesterol levels in MDA-MB-231 cells at selected concentrations of HPβCD at 1 mM, 5 mM, 10 mM, 20 mM, 50 mM and negative samples. Plumbagin was used as a positive control. Data are mean±standard deviation S.D. (n=3) from raw data, where *p<0.05, **p<0.01 and ***p<0.001 significant difference to untreated control.

FIG. 24: Representative graph comparing the total, free and esterified cholesterol levels in MRC-5 cells at selected concentrations of HPβCD at 1 mM, 5 mM, 10 mM, 20 mM, 50 mM and negative samples. Plumbagin was used as a positive control. Data are mean±standard deviation S.D. (n=3) from raw data, where *p<0.05, **p<0.01 and ***p<0.001 significant difference to untreated control.

FIG. 25 A to C: Pictures taken on fluorescent microscope (The FLoid® cell imaging station) after Filipin staining, comparing the overall cholesterol levels in MCF7 cells. Prominent cells are marked with arrows. (A) shows Fluorescence in untreated cells, (B): Fluorescence in positive sample (5 mM MBCD) and (C) Fluorescence in 10 mM HPβCD treated cells.

FIG. 26 A to C: Pictures taken on fluorescent microscope (The FLoid® cell imaging station) after Filipin staining, comparing the overall cholesterol levels in MDA-MB-231 cells. Prominent cells are marked with arrows. (A): Fluorescence in untreated cells, (B): Fluorescence in positive sample (5 mM MBCD) and (C): Fluorescence in 10 mM HPβCD treated cells.

FIGS. 27 A and B: Western blot assay to detect protein expression of SREBP-1 in MCF7 cells. (A): Protein levels in untreated cells, (B): Protein levels in HPβCD treated cells (10 mM HPβCD). β-tubulin was used as a loading control.

FIGS. 28 A and B: Western blot assay to detect protein expression of SREBP-1 in MDA-MB-231 cells. (A): Protein levels in untreated cells. (B): Protein levels in HPβCD treated cells (10 mM HPβCD). β-tubulin was used as a loading control.

FIGS. 29 A and B: Western blot assay to detect protein expression of SREBP-1 in MRC-5 cells. (A): Protein levels in untreated cells, (B): Protein levels in HPβCD treated cells (10 mM HPβCD). R-tubulin was used as a loading control.

FIG. 30 A to C: Mice images post euthanization showing tumour sizes in untreated group at a late stage, injected with MDA-MB-231 cells. (A): Mice post euthanization, (B): Harvested tumours and (C): Tumour sizes.

FIG. 31 A to C: Mice images post euthanization showing tumour sizes in treated group at a late stage, injected with MDA-MB-231 cells and treated with HPβCD (3000 mg/kg b.w.) for 10 doses. (A): Mice post euthanization, (B): Harvested tumours and (C): Tumour sizes. Results show tumour reduction by 73.9% compared to the untreated group.

FIG. 32 A to C: Mice images post euthanization showing tumour sizes in treated group at an intermediate stage, injected with MDA-MB-231 cells and treated with HPβCD (3000 mg/kg b.w.) for 10 doses. (A): Mice post euthanization, (B): Harvested tumours and (C): Tumour size. Results show tumour reduction by 94% compared to the untreated group.

FIGS. 33 A and B: Mice images showing tumour sizes in untreated group at an early stage, injected with MDA-MB-231 cells (A): Initiation of tumour and (B): Tumour sizes

FIGS. 34 A and B: Mice images showing tumour sizes in treated group at an early stage, injected with MDA-MB-231 cells and treated with HPβCD (3000 mg/kg b.w.) for 10 doses (A): Termination of tumour and (B): Complete healing of tumour.

FIG. 35: All three healed mice (No. 2, 4 and 7) tested for relapse of ER− tumour for a period of 4 weeks. Results show no relapse at all.

FIG. 36 A to F: Shows hematoxylin and cosin staining and examination of untreated, intermediate and late in MDA-MB-231 mice. (A) Shows untreated, wherein tumour cells are seen to dissect between skeletal muscle fibers (arrows) in the photomicrograph (magnification at ×100). (B) Shows untreated, wherein an area of necrosis is identified (arrow) (magnification at ×100). (C) Shows intermediate, wherein numerous apoptotic cells are identified (arrows)(magnification ×200). (D) Shows intermediate, wherein a number of mitotic figures in the micrograph (magnification ×200). (E) Shows late stage, wherein numerous apoptotic cells are identified (arrows) (magnification ×200). (F) Shows late stage, wherein a large area of geographic necrosis (asterisk *) is present together with scattered necrotic foci (arrows) (magnification ×100)).

FIG. 37: Graphical representation of ALT/AST levels in all 14 mice across the study. Permissible range in mice, for ALT is 28-132 U/L and AST is 59-247 U/L. Data are mean w standard deviation S.D. (n=3 or n=4) from raw data, where *p<0.05, and ‘ns’ corresponds to not significant difference to untreated control.

FIG. 38: List of genes up (red) and down-regulated (green) in human lipoprotein signalling. Results in MDA-MB-231 particularly, shows upregulation of ABCA1 gene (highlighted) suggesting increased efflux of cholesterol and phospholipids to lipid-poor apolipoproteins (apoA1 and apoE), which then form nascent high-density lipoproteins (HDL) upon treatment with HPβCD. In black and white drawings an asterisk * represented upregulated and a double asterisk ** represents down regulated.

FIG. 39: List of genes up and down-regulated in human breast cancer, where ‘red’ represents upregulated and ‘green’ represents downregulated genes. In black and white drawings an asterisk * represented upregulated and a double asterisk ** represents down regulated.

FIG. 40: Cytoscape analysis of genes up and down-regulated in human lipoprotein signalling (MDA-MB-231). Green to orange represent low to high levels of expression, respectively. In black and white drawings orange is represented by an asterisk * and green by a double asterisk **.

FIG. 41: Cytoscape analysis of genes up and down-regulated in human breast cancer (MDA-MB-231). Green to orange represent low to high levels of expression, respectively. In black and white drawings orange is represented by an asterisk * and green by a double asterisk **.

FIG. 42: Cytoscape analysis of genes up and down-regulated in human lipoprotein signaling (MCF7). Green to orange represents low to high levels of expression. In black and white drawings orange is represented by an asterisk * and green by a double asterisk **.

FIG. 43: Cytoscape analysis of genes up and down-regulated in human breast cancer (MCF7). Green to orange represents low to high levels of expression, respectively. In black and white drawings orange is represented by an asterisk * and green by a double asterisk **.

FIG. 44: shows HPβCD is capable of binding human proteins Shown are the representative SPR sensorgrams representing direct interaction of HPβCD with (A) ABCA1: (B) ADAM3; (C) AKT1; (D) GATA3: (E) Cathepsin and (F) SFRP1. The sensorgrams were analysed to generate the kinetics in Table 3.

FIG. 45(A) to (D): (A) to (C) show Filipin staining (for cholesterol) of mice sections. (A) 4 from untreated, (B) 3 from intermediate and (C) 4 from late stage. Results show decreased cholesterol in intermediate and late stage tumours compared to untreated as seen in (D).

FIG. 46 (A) to (D): (A) to (C) shows Filipin staining of MDA-MB-231 cells for cholesterol. Results show decreased cholesterol in treated cells compared to untreated. (A) Shows untreated, (B) shows positive, (C) shows treated, and (D) shows a graphical representation showing decreased cholesterol in treated cells compared to untreated cells.

FIG. 47 (A) to (D): (A) to (C) shows BODIPY staining of MDA-MB-231 cells for lipid droplets. Green shows lipid staining while blue is the nuclear stain. Results show decrease in lipid droplets in treated cells compared to untreated. Lipid droplets store cholesterol and HPβCD was able to reduce accumulation and storage of cholesterol in cancer cells. (A) Shows untreated. (B) shows positive. (C) shows treated, and (D) shows a graphical representation showing decreased cholesterol in treated cells compared to untreated cells.

FIG. 48 (A) to (D): (A) to (C) shows ALEXA FLUOR staining of MDA-MB-231 cells for lipid raft dissociation. Results show decrease in lipid rafts in treated cells compared to untreated as graphically shown in (D).

FIG. 49 (A) to (D): (A) to (D) show photographs which shows development of tumours in 4 mice (ER+). (A) shows Mouse No. 29 tumour 173.21 mm³. (B) shows Mouse No. 30 tumour 163.46 mm³. (C) shows Mouse No. 31 tumour 188.01 mm³, and (D) shows Mouse No. 32 tumour 103.74 mm³. Average tumour size 157.1 mm³.

FIG. 50(A) to (D): (A) to (D) shows photographs of ER+ Mice treated with HPβCD for 5 weeks. (A) shows Mouse no. 29 wherein the tumour healed completely. (B) shows Mouse No. 32 wherein the tumour healed completely, (C) shows Mouse No. 31 wherein the tumour reduced to 21.28 mm³, and (D) shows Mouse No. 30 wherein the tumour healed completely. Avery tumour size 5.3 mm³. Tumour reduction 96.6%

FIG. 51 (A) to (D): (A) to (C) shows Filipin staining of MCF7 cells for cholesterol, wherein (A) is untreated. (B) is positive and (C) is treated. (D) graphically shows decreased cholesterol in treated cells when compared to untreated cells.

FIG. 52(A) to (D): (A) to (C) shows BODIPY staining of MCF7 cells for lipid droplets. Green (darker areas in the black and white figures) shows lipid staining while blue (lighter defined dots in black and white figures) is the nuclear stain. (A) is untreated, (B) is positive and (C) is treated. (D) shows graphically the results wherein decrease in lipid droplets in treated cells compared to untreated. Lipid droplets store cholesterol and HPβCD was able to reduce accumulation and storage of cholesterol in cancer cells.

FIG. 53 (A) to (D): (A) to (C) shows ALEXA FLUOR staining of MCF7 cells for lipid raft dissociation, wherein (A) is untreated, (B) is positive and (C) is treated. (D) shows graphically the results wherein a decrease in lipid rafts in treated cells compared to untreated.

### DETAILED DESCRIPTION OF THE INVENTION

The Summary of the invention, including all first to further aspects is repeated hereunder bv way of reference only to avoid repetition. Specific, but non-limiting embodiments of the invention will now be described.

Generally, in accordance with a first aspect of this invention there is provided 2-hydroxypropyl-β-cyclodextrin (HPβCD) for use in the treatment of breast cancer in a human or animal body. Preferably the breast cancer is triple negative breast cancer. The Applicant has surprisingly and unexpectedly found that HPβCD may treat, ameliorate and/or prevent solid tumor cancers, particularly breast cancer, and more particularly triple negative breast cancer. This is particularly surprising and unexpected since cyclodextrins are typically no more than excipients in pharmaceutical compositions and are typically inactive and/or inert. This surprising and unexpected effect is further advantageous since cyclodextrins and particularly HPβCD is safe for human and animal use. Moreover, the cost implications of developing a pharmaceutical drug comprising HPβCD for the treatment of triple negative breast cancer will be comparatively low. Without being limited to theory, the Applicant believes that HPβCD extracts extra cholesterol from breast cancer cells, thus depriving said breast cancer cells of basic fuel for cell division and proliferation causing cell death, as supported by the experimental data herein.

Further experimental protocols to develop dosage regimes will also be conducted in time. Typically, the amount of HPβCD for administration to the human or animal in need thereof is linked to, but not limited to, body weight, tumor size, tumor weight and/or whether or not the cancer has metastasized.

Typically, when in use to treat breast cancer the 2-hydroxypropyl-β-cyclodextrin (HPβCD) is formulated as a pharmaceutical composition.

The pharmaceutical composition may be for administration via a parenteral and/or non-parenteral route.

Parenteral administration may include, but is not limited to, intravenous, subcutaneous, intramuscular, or implantation into the human or animal body.

Non-parenteral administration may include, but is not limited to, oral, rectal, vaginal, sublingual, buccal, and intranasal delivery of the pharmaceutical composition into the human or animal body.

The pharmaceutical composition may include an excipient. It is to be understood that other components such as fillers, taste masking agents, colorants, vitamins, minerals, other active pharmaceutical ingredients (APIs) and the like may also be included into the composition.

The pharmaceutical composition may include further cyclodextrins from the family of cyclodextrins.

The invention extends to the second, third, fourth and fifth aspects of this invention as herein described.

### Examples

The Examples herein below are not intended to limit the scope of this invention in any way, and are provided merely to exemplify and/or illustrate certain embodiments of the invention.

The cytotoxic activity of HPβCD was tested in four different cell lines which constituted the in vitro studies. These four cell lines were: Estrogen positive (ER+) MCF7 breast cancer cells. Triple negative MDA-MB-231 breast cancer cells, MRC-5 (Normal Human Lung Fibroblasts) and HEK-293 (normal human embryonic kidney cells). Cytotoxic, apoptosis and cholesterol based assays were performed in vitro. The results are presented below. In vivo studies (mice model) are also presented, and further studies for both ER+ and triple negative breast cancer cells are envisaged. Based on preliminary investigations (in vitro and in vivo) a comprehensive mechanism of action will be elucidated.

Cells were cultured in appropriate medium supplemented with 10% FCS, penicillin (100 U/ml), and streptomycin (100 μg/ml) (Gibco) at 5% CO2 in a 37° C. incubator.

### In Vitro Studies:

### Growth Inhibition Assays—MTT Assay

Four different cell lines were used MCF7 (ER+). MDA-MB-231 (ER−), MRC-5 (Lung Fibroblasts) and HEK-293 (Embryonic kidney). MRC-5 and/or HEK-293 were used as control cell lines depending on availability.

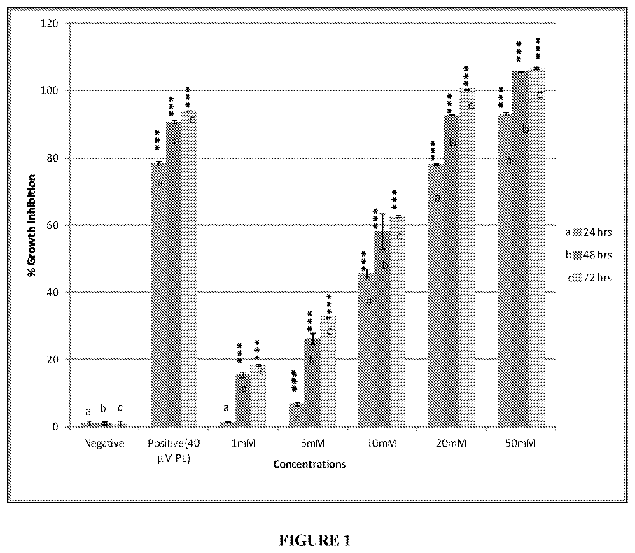

In order to determine whether HPβCD has the ability to induce cancer cell death a primary screening using an MTT assay was performed. Three different time points 24 hrs. 48 hrs & 72 hrs treatment were selected in order to establish the potency of HPβCD. The results are shown in FIGS. 1 to 4.

TABLE 1
Shows IC₅₀ values for MCF7, MDA-MB-
231, MRC-5 and HEK-293 cells.

| | IC₅₀ Values | | |
|---|---|---|---|
| Cell Line | 24 hrs | 48 hrs | 72 hrs |
| MCF7 | 11.11 mM | 9.82 mM | 7.45 mM |
| MDA-MB-231 | 12.34 mM | 11.22 mM | 8.56 mM |
| MRC-5 | 20.67 mM | 19.89 mM | 41.69 mM |
| HEK-293 | 22.81 mM | 26.53 mM | 50.26 mM |

Results obtained, showed ˜45% growth inhibition at 10 mM concentration of HPβCD. Subsequently, the percentage growth inhibition kept on increasing with an increase in concentrations of HPβCD. The results hold true for all the three time points, thus indicating its potential ability, without being limited to theory, to disrupt the mitochondrial processes and its probable slow action (FIG. 1). 40 μM Plumbagin (PL) was used as a positive control and showed almost 80%, 90% & 100% growth inhibition at 24 hrs, 48 hrs & 72 hrs respectively. Plumbagin is a well-known compound proven to inhibit cancer cell growth.

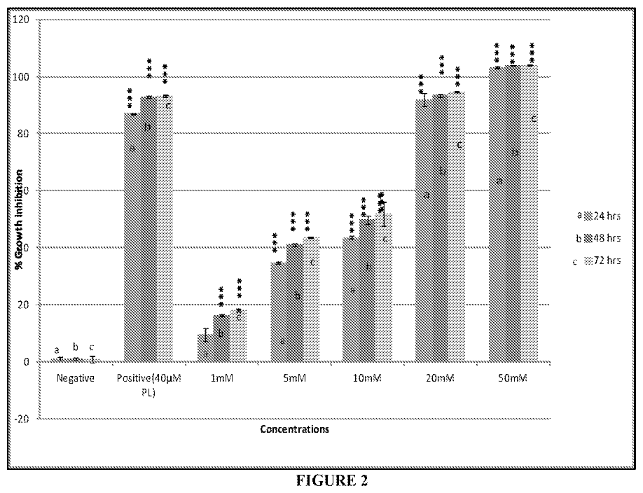

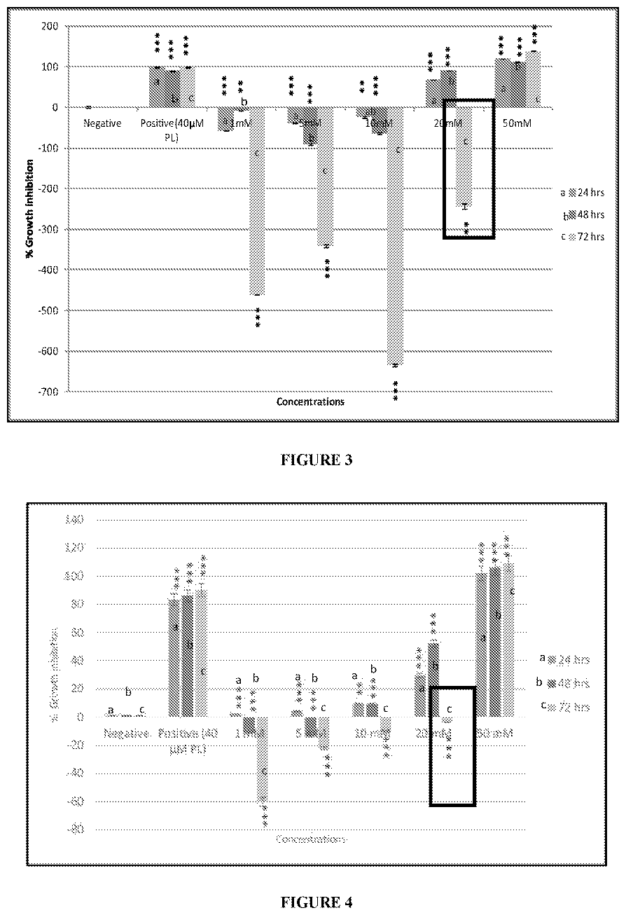

Like MCF7s three different time points 24 hrs. 48 hrs & 72 hrs were selected for MDA-MB-231 cells. Results obtained, showed ˜45% growth inhibition at 10 mM concentration of HPβCD and again, there was a gradual increase in the percentage growth inhibition as the concentrations of HPβCD increased. The results were similar for all the three time points, thus indicating the fact that HPβCD is quite effective across different breast cancer cells (FIG. 2). 40 μM Plumbagin (PL) was used as a positive control and showed almost 85%, 92% & 95% growth inhibition at 24 hrs, 48 hrs & 72 hrs respectively.

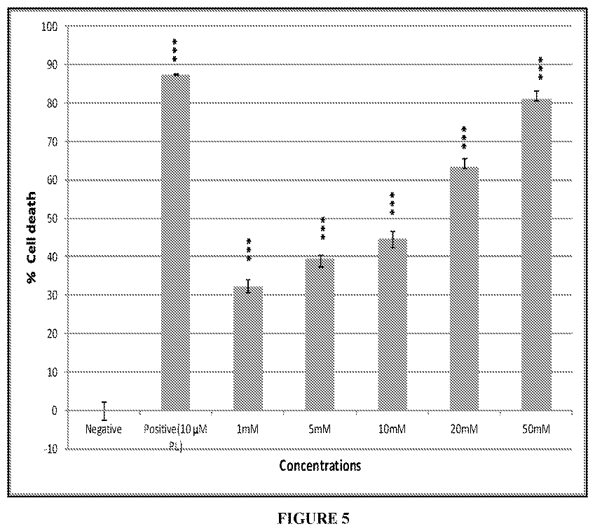

For the control cell lines MRC-5 & HEK-293 similar time points were selected (24 hrs, 48 hrs. 72 hrs) to maintain consistency. Results obtained, showed absolutely zero growth inhibition till 10 mM concentration of HPβCD. The results hold true for two time points (24 hrs and 48 hrs). Interestingly, at 72 hrs (Black box in FIG. 3 & FIG. 4), even 20 mM became non-toxic to the cells, thus indicating that HPβCD is actually assisting the cells to survive and all the unhealthy cells are reprogramming themselves and behaving normally. 50 mM was seen to be very toxic for the cells (FIG. 3 & FIG. 4) because the dosage was too high. Every cell has a threshold value for a particular drug concentration beyond which it becomes toxic and every drug follows a dose-response approach. Upon calculating the IC₅₀ values which is the concentration required to cause 50% growth inhibition, it was seen that almost double the concentration of HPβCD was required to cause 50% growth inhibition in both the cell lines at 24 hrs (Table 1) and at 72 hrs the concentration went up to almost five times as compared to the cancerous cells, thus indicating that HPβCD is specifically toxic to cancerous cells.

### Cell—APOPercentage Apoptosis Assay

APOPercentage assays gives a measure of overall apoptosis in the cell. Apoptosis is an early event and therefore just one time point (24 hrs) was selected and tested on four cell lines MCF7, MDA-MB-231, MRC-5 & HEK-293 cells. 10 μM Plumbagin was used a positive control as opposed to 40 μM from MTT because higher concentrations of plumbagin was way too toxic for the cells and to capture the early activity, optimal dosage was required.

To confirm the growth inhibition obtained from the MTT assay was actually due to apoptosis, a Cell-APOPercentage Apoptosis assay was performed according to known methods.

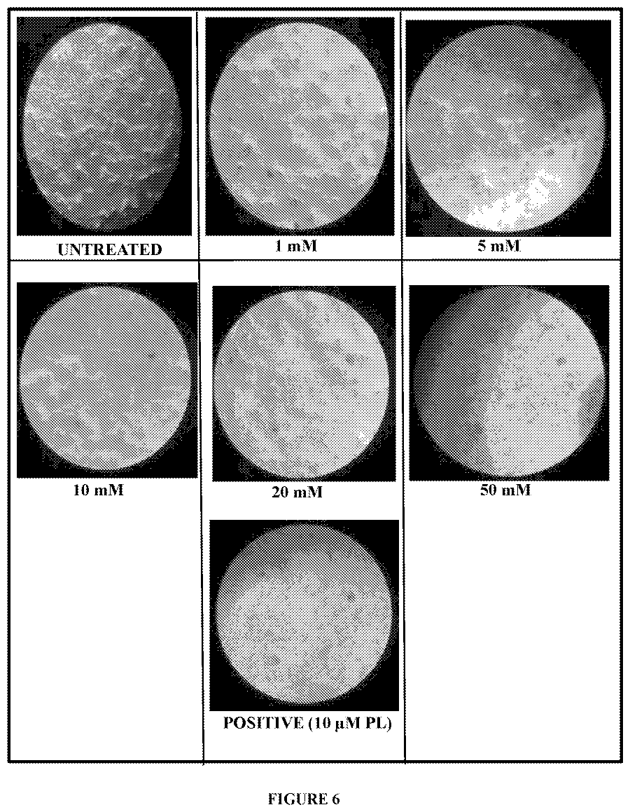

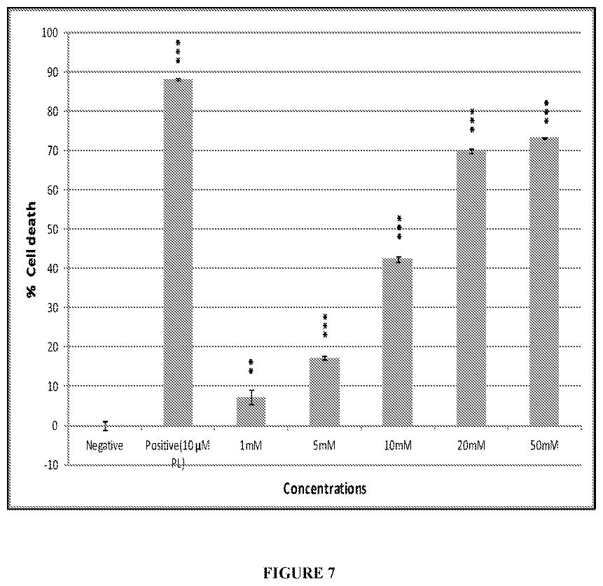

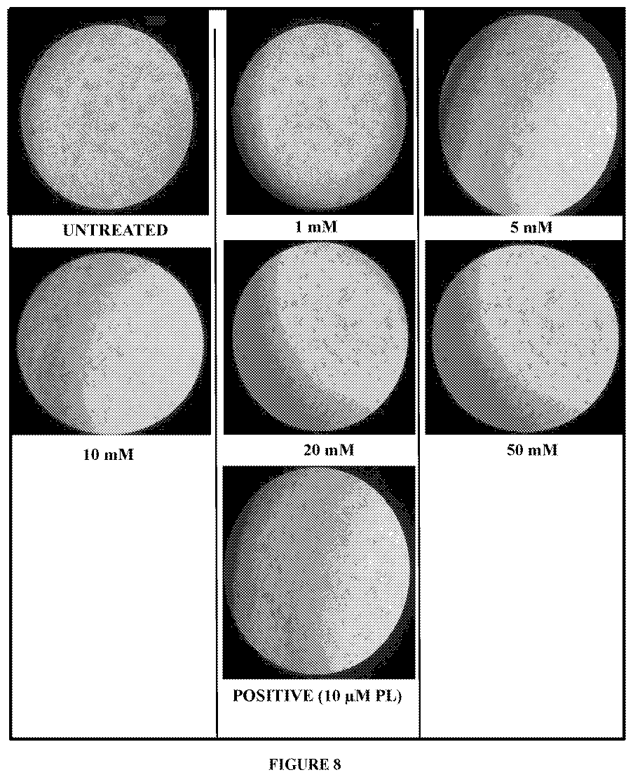

Results for MCF7 gave us around 45% cell death at 10 mM concentration of HPβCD. The cell death percentage kept on increasing with an increase in concentration of HPβCD across all the treatments (FIG. 5). 10 μM Plumbagin was used as a positive control and yielded almost 90% apoptosis. Pictures (FIG. 6) taken under a light microscope post staining with the APOPercentage dye validates our graphical data where it can be clearly seen that as the concentration of HPβCD increases the pink intensity (in colour versions of the figures) of the dye keeps on increasing thus indicating an increase in apoptosis. In the black and white images the number of defined dark grey spots relative to the background increases with the as the concentration of HPβCD increases indicating an increase in apoptosis.

For MDA-MB-231 cells the results obtained gave us around 40% cell death (lesser than MCF7) at 10 mM concentration of HPβCD (FIG. 7). Like MCF7's, the cell death percentage kept increasing at all the higher concentrations of HPβCD and the maximum cell death achieved was ˜70% at 50 mM HPβCD. 10 μM Plumbagin was used as a positive control and the percentage of apoptosis observed was almost 90%. Images taken under alight microscope post staining with the APO Percentage dye validates our graphical data (FIG. 8) where, like MCF7's we can clearly see that with an increase in HPβCD concentration, the pink intensity (in colour versions of the figures) of the dye kept increasing thus indicating a gradual progression in apoptosis. In the black and white images the number of defined dark grey spots relative to the background increases with the as the concentration of HPβCD increases indicating a progression in apoptosis.

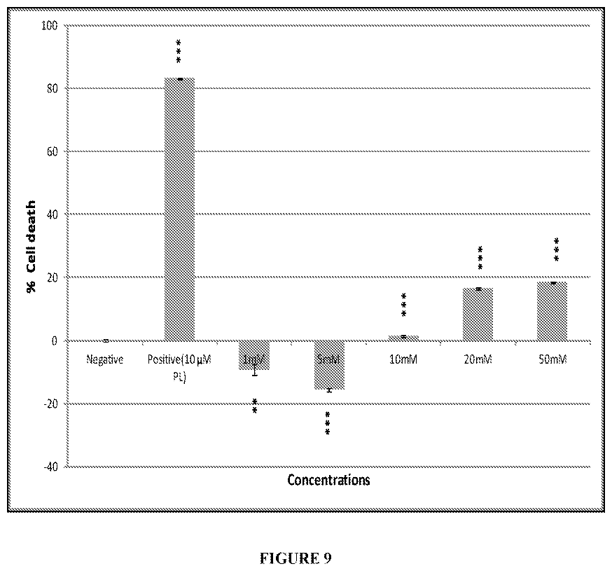

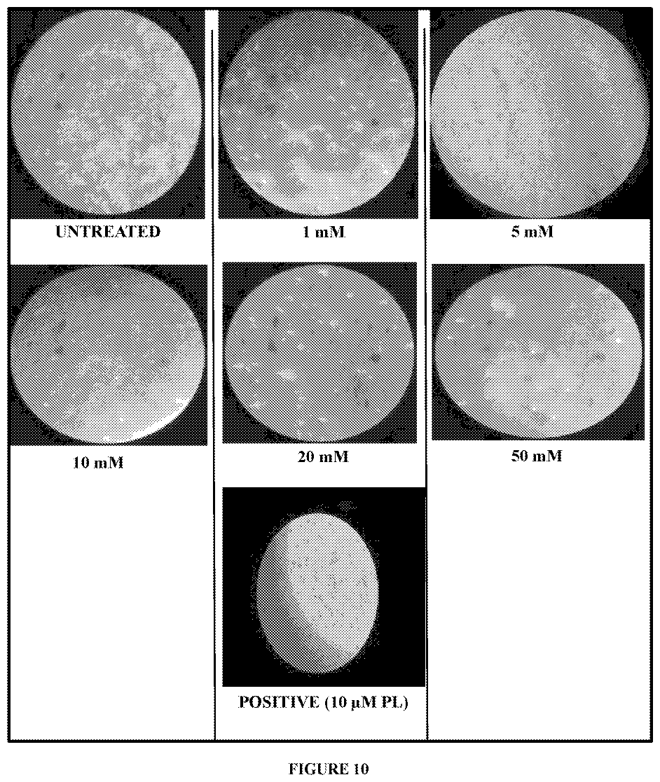

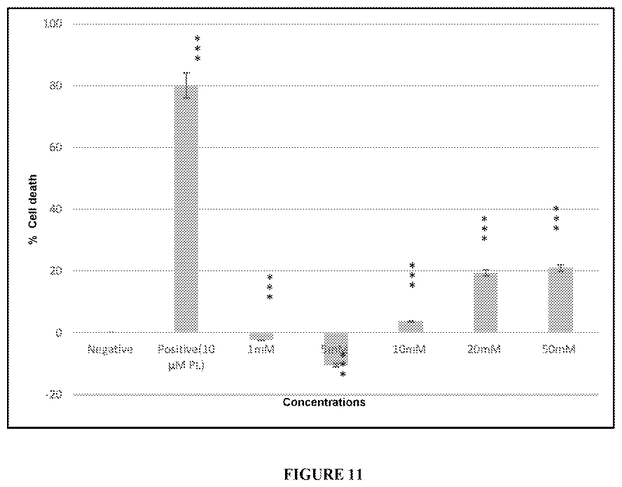

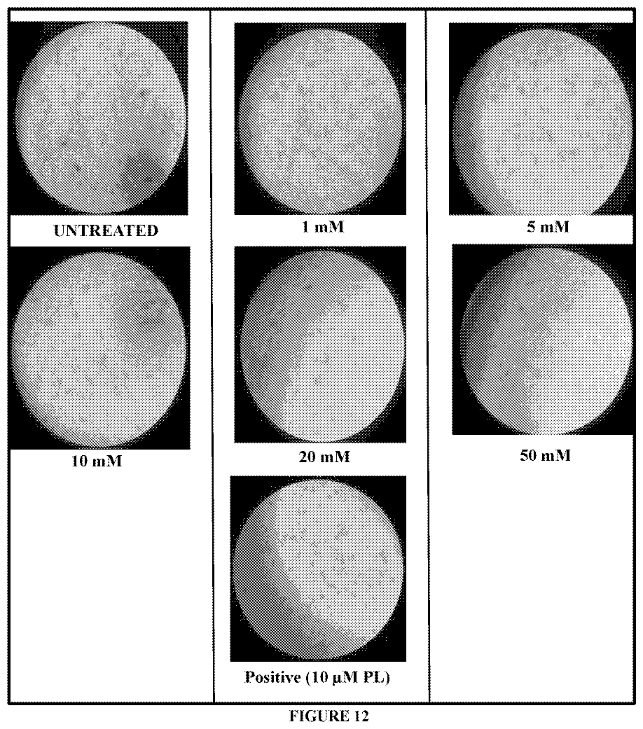

Finally, for the normal cells (MRC-5 & HEK-293), the results obtained approximated almost zero percentage cell death till 10 mM concentration of HPβCD (FIG. 9). 20 mM & 50 mM gave us ˜15% and ˜18% cell death respectively for both the cell lines. Pictures taken under a light microscope post staining with the APO Percentage dye validated our graphical data (FIG. 10) and it is quite evident that there was no dye uptake by the cells till the concentration of 10 mM HPβCD. At higher concentrations, we do see very little pink intensity (in the colour versions of the figures) which corroborates with our graphical data and therefore confirms the fact that HPβCD is indeed toxicologically benign to normal cells. In the black and white images the number of defined spots relative to the background does not increase very much. See FIGS. 11 and 12 for HEK-293 cell lines response to HPβCD showing a similar situation as MRC-5.

### Mitochondrial Outer Membrane Potential Assay (MOMP)

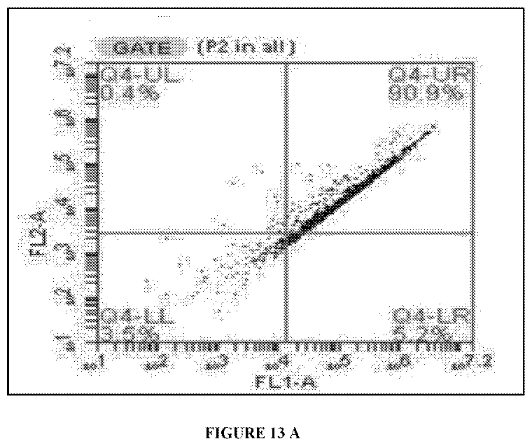

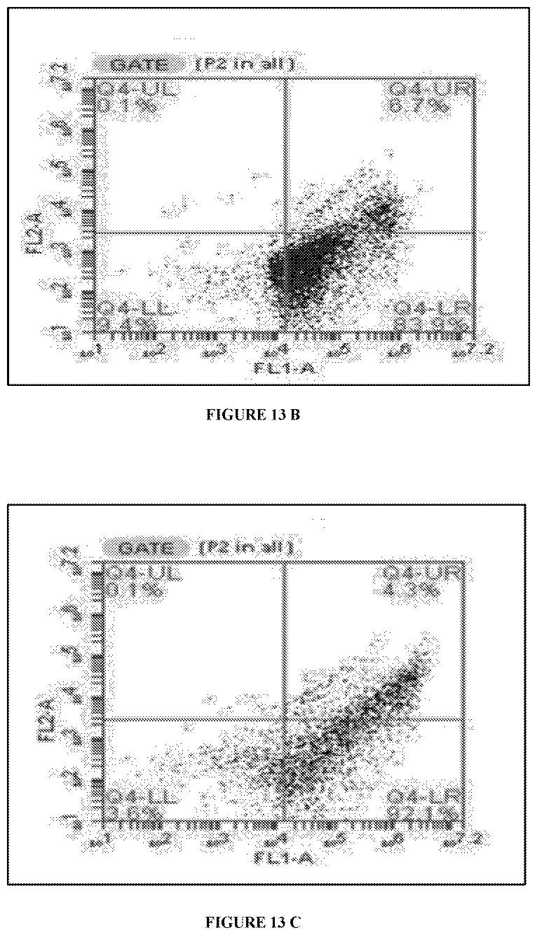

One of the key hallmarks of apoptosis is the loss of mitochondrial membrane potential. To investigate that aspect we did a MOMP assay which validates our Cell-APOPercentage assay. This assay was tested on three cell lines MCF7, MDA-MB-231 & MRC-5. The top right and bottom hand quadrants of each plot represents healthy (FL2-A) and apoptotic (FL1-A) cells respectively (see FIGS. 13 to 15).

From the results obtained from MTT (IC₅₀ values) and Cell-APOPercentage assays it was quite evident that ˜10 mM HPβCD caused cellular growth inhibition through apoptosis in cancer cells and had little or no effect in normal cells. Therefore, we selected just 10 mM HPβCD going forward as the only working concentration for HPβCD treatment. 24 hrs time point was selected (HPβCD & 10 mM H₂O₂) because mitochondrial membrane potential loss is an early cellular event.

Results obtained for MCF7 (FIG. 13) showed 92.1% apoptotic population at 10 mM concentration of HPβCD (treated), whereas the healthy population in untreated cells was 90.0%. The percentage of apoptotic cells in the positive sample (10 mM H₂O₂) was 92.1%. H₂O₂, like PL is an agent well-known to cause apoptosis.

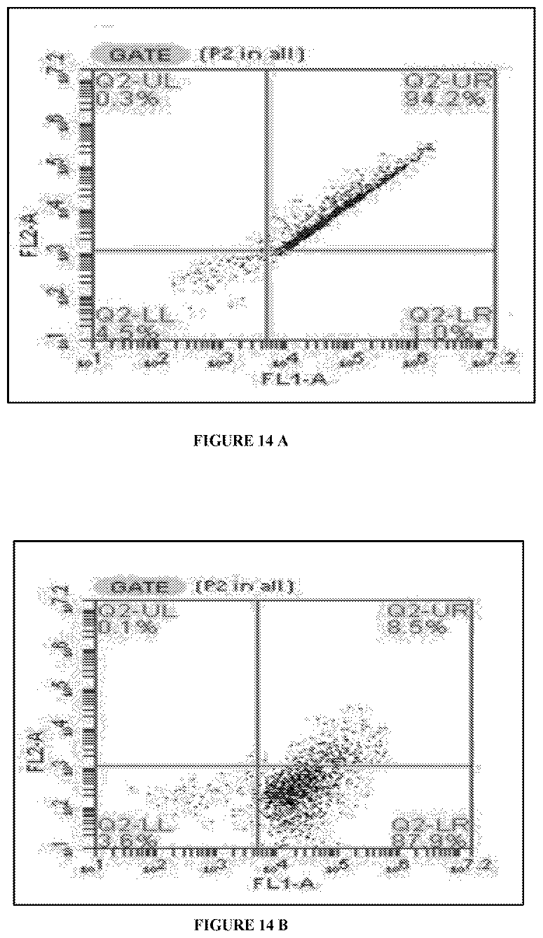

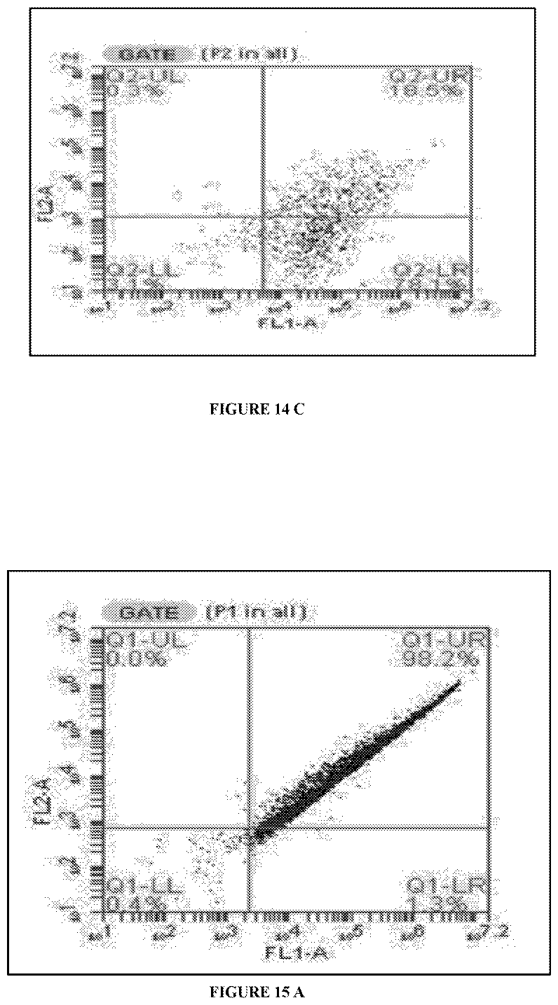

MDA-MB-231 cells gave a 78.1% cell death at 10 mM concentration of HPβCD (FIG. 14), whereas the healthy the population in untreated was still quite high at 94.2%. The positive sample (10 mM H₂) as expected yielded a very high 92.4% apoptotic cells.

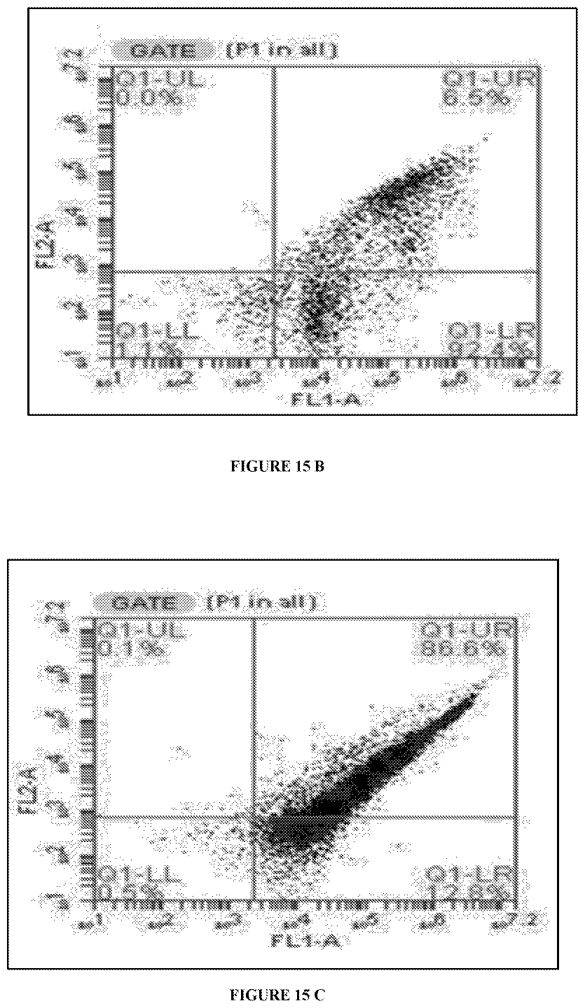

For the normal cells (MRC-5), results obtained gave us 98.2% non-apoptotic/healthy population and 12.8% apoptotic cells at 10 mM concentration of HPβCD (FIG. 15). Thus, confirming that HPβCD is indeed quite non-toxic to normal cells, whereas being significantly apoptotic to cancerous cells via the intrinsic apoptosis pathway and causing loss in mitochondrial membrane potential. This is a most surprising and unexpected result.

### ROS Assay (Reactive Oxygen Species)

To further validate the occurrence of apoptosis and to investigate the exact mechanisms if apoptosis, a ROS assay was conducted to capture the production of ROS. This is because generation of ROS has also been shown to be a characteristic feature in apoptosis. In order to be in solidarity with this fact, a ROS assay was performed in three cell lines: MCF7, MDA-MB-231 & MRC-5. Like before, HPβCD and the positive (10 mM H₂O₂) were treated for 24 hrs. The right hand section in each plot represents the production of ROS.

Cancer cells in general have enhanced metabolism due to the fact that they divide rapidly as compared to normal cells thereby leading to an abundant generation of ROS.

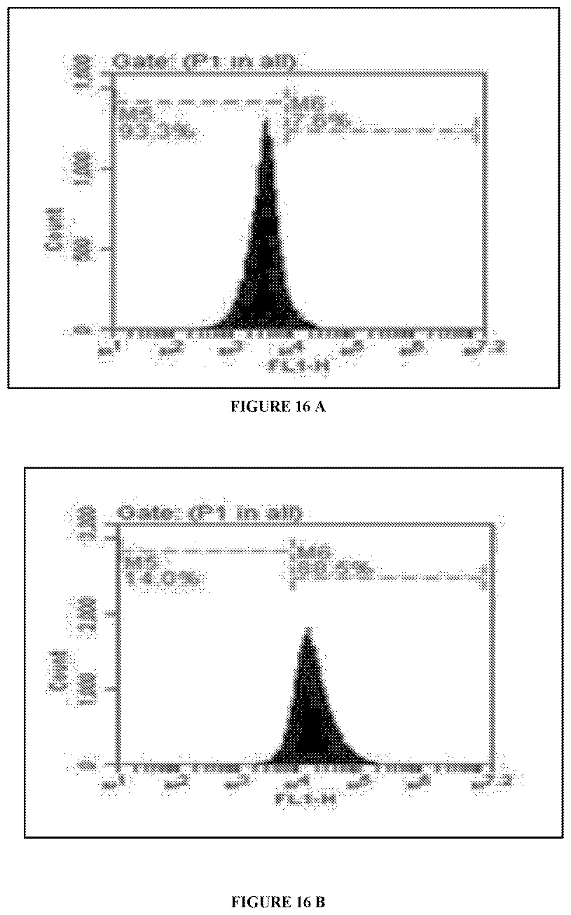

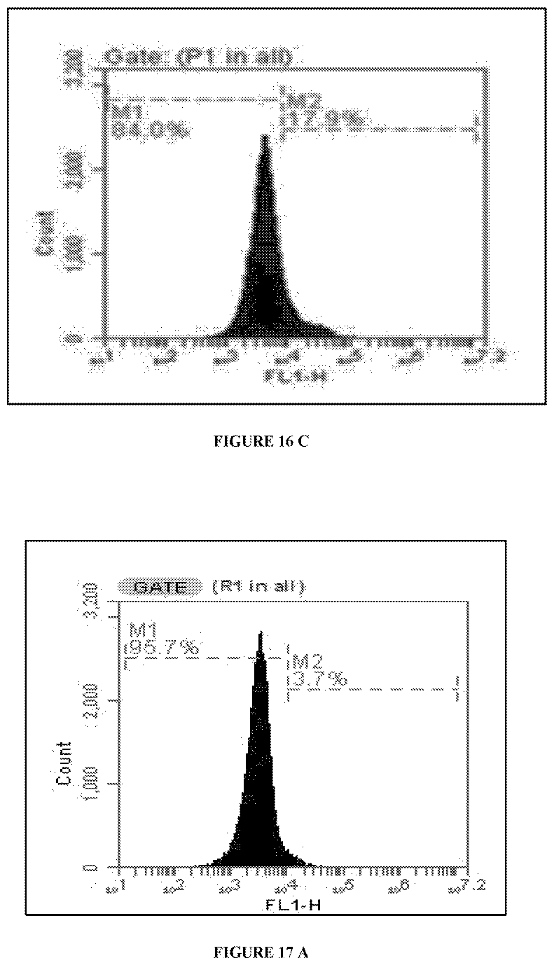

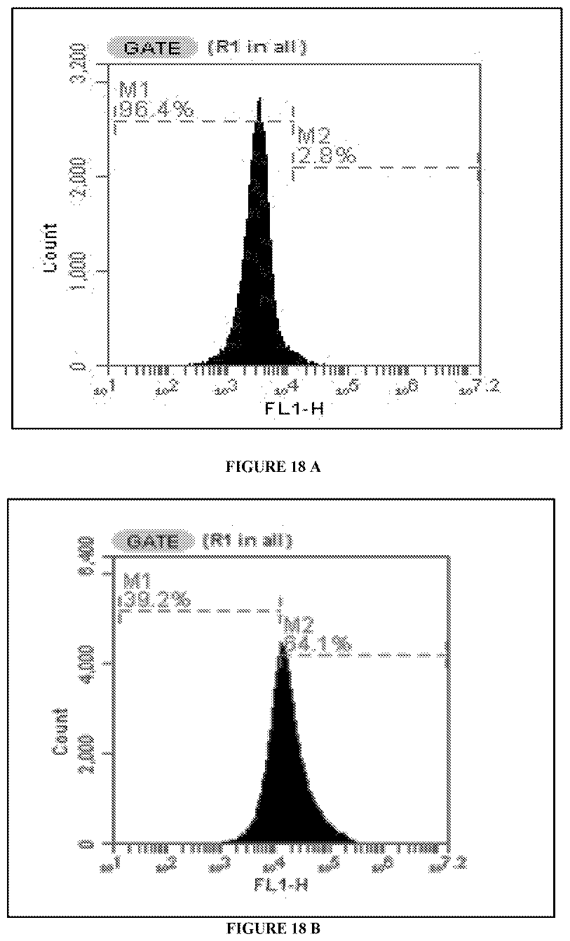

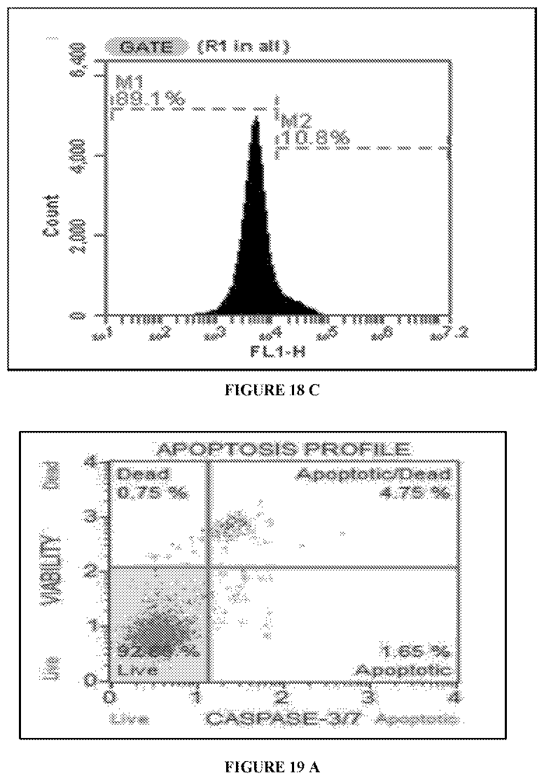

From FIG. 16 C, it can be seen that the percentage of ROS production in MCF7 cells for 10 mM HPβCD treatment was 17.9% whereas in the untreated it was 7.6% (FIG. 16 A). Although this was about 2.5 fold more as compared to the untreated sample but was not too significant especially if we look at the ROS production in the positive sample which had a very high 88.5% ROS generation (FIG. 16 B) and also the levels of ROS generation in normal cells (MRC-5) which showed 10.8% ROS activity at 10 mM HPβCD treatment (FIG. 18 C).

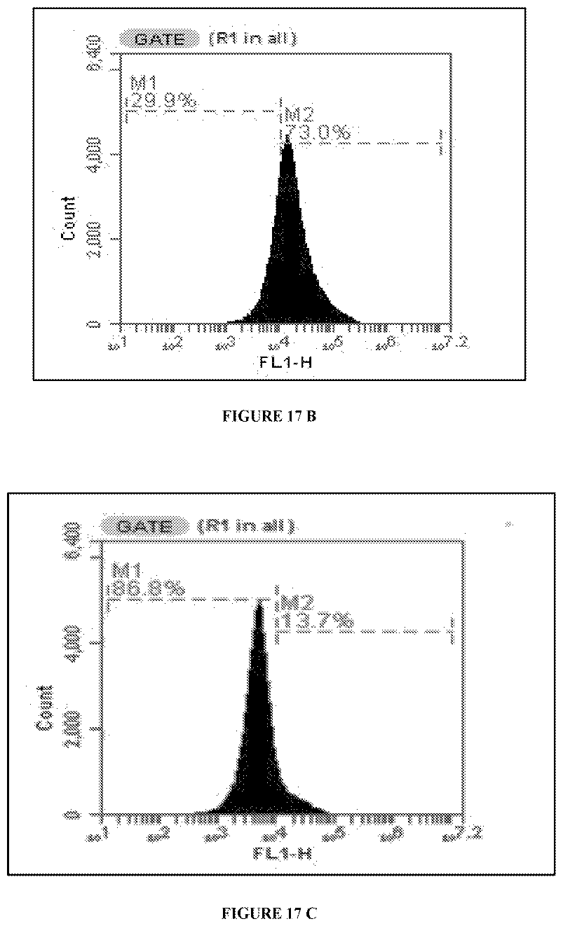

MDA-MB-231 cells gave similar results like MCF7's and the percentage of ROS production for 10 mM HPβCD treatment was 13.7% (FIG. 17 C) whereas in the untreated it was 3.7%. Again, this was about 4 fold more as compared to the untreated cells but not too staggering when we look at the positive sample (FIG. 17 B) which showed 73% ROS production and in normal cells where 10.8% ROS production was seen at 10 mM HPβCD treatment (FIG. 18 C).

MRC-5 cells in general showed 2.8% ROS activity in untreated cells (FIG. 18 A) and like MCF and MDA-MB-231 cells the ROS production went up to almost 4 folds at 10 mM HPβCD treatment (10.8%). Thus, it was inferred that although HPβCD does cause apoptosis but it doesn't do so via enormous production of ROS. Downregulation of ROS in apoptosis has often been observed and it can happen considering that ROS has significant roles in physiological processes. It is quite possible there is an over expression of detoxifying enzymes like catalase and glutathione reductase, which are responsible for the maintenance of free radicals. However, further studies are required to elucidate an exact mechanism.

### CASPASE 3/7 Assay

The final step in apoptosis following the intrinsic pathway is the activation of executioner caspases like caspase 3 and caspase 7 which leads to the release of Cytochrome c. Therefore, to confirm that cell death was due to the mitochondrial mediated pathway of apoptosis a Caspase 3/7 assay was conducted.

Caspases are activated in response to a myriad of cellular death stimuli and they dismantle the cell. Caspase 3 and caspase 7 contribute to the majority of proteolytic cleavage occurring in apoptosis. As a final step to confirm and validate apoptosis via the mitochondrial intrinsic pathway, the Caspase 3/7 assay was conducted on three cell lines: MCF7, MDA-MB-231 & HEK-293. HPβCD and positive (40 μM PL) were treated for 24 hrs. The percentage of dead/apoptotic cells are clearly indicated in specific corners of each quadrants in the above plots.

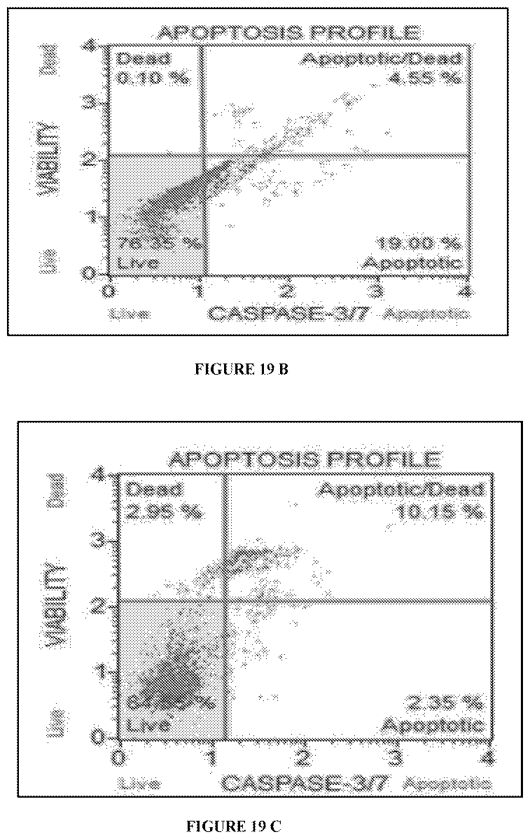

For MCF7 cells, the total apoptosis observed in untreated cells were 6.4% (FIG. 19 A) whereas in the treated cells (10 mM HPβCD) it went up to 12.5% (FIG. 19 C) which was almost two times more as compared to the untreated sample. For the positive sample (40 μM PL), the total apoptotic population was 23.55% (FIG. 19 B). The overall low caspase 3/7 activity was because MCF7 cells do not express caspase 3 activity in general, and this is well reported. Because this particular assay gives a measure of both caspase 3 and 7 therefore even if caspase 7 activity was quite high, the overall measurement of apoptosis was neutralized by the absence of caspase 3 activity. So, the cell death activity in MCF7 was solely due to caspase 7 and quite possibly, caspase 7 activity in MCF7 cells was quite low overall.

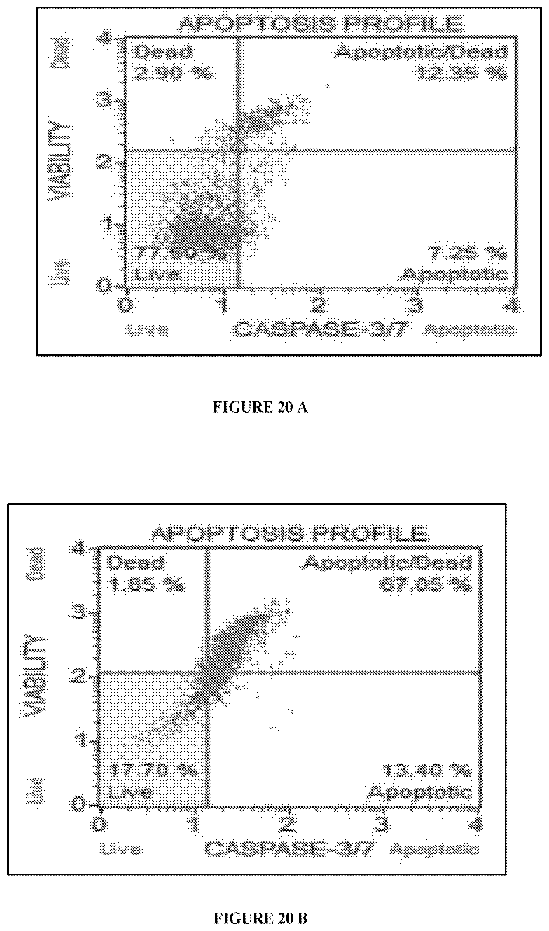

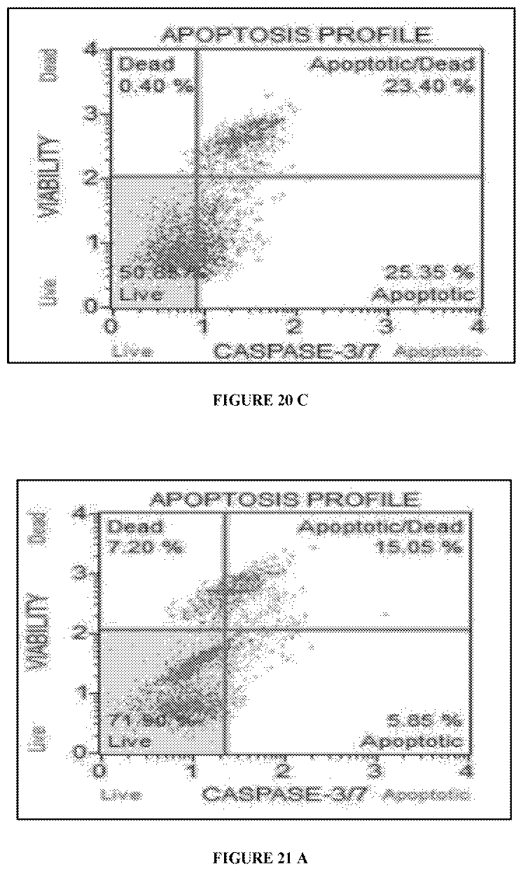

MDA-MB-231 cells on the other hand showed a better activity of Caspase 3/7. In the untreated cells the total apoptotic population was seen to 19.6% (FIG. 20 A) which went up to 48.75% in 10 mM HPβCD treated sample (FIG. 20 C). The aforementioned confirming that in MDA-MB-231 cells, HPβCD does act via the intrinsic pathway and causes a disruption in the mitochondria thereby activating the executioner caspases 3 and caspase 7. In the positive sample (40 μM PL), the total apoptotic population was 80.45% (FIG. 20 B).

In the normal cells (HEK-293), the total healthy population in the untreated cells was 71.90% (FIG. 21 A), which decreased only partially in the 10 mM HPβCD treated sample (FIG. 21 C) and went down to 66.75%. In the positive sample (40 μM PL), the total apoptotic population was 72.4% (FIG. 21 B). This now absolutely warrants that HPβCD is indeed non-toxic to normal cells. HPβCD's non-toxicity was later further validated in our in vivo assays.

### Cholesterol Quantification Assay

To investigate if the apoptosis observed from all the above assays was due to cholesterol depletion, a cholesterol assay was performed on all three cell lines: MCF7, MDA-MB-231 and MRC-5. HPβCD and the positive PL, a known cholesterol depletor were treated for 24 hrs.

It is a well-known fact that cancer cells, have elevated levels of cholesterol since cholesterol is an integral part of the cell membrane and is recruited by the cancer cells for rapid division.

HPβCD and the positive (40 μM PL) were treated for 24 hrs. This assay gave us an estimate of total cholesterol, cholesterol esters and free cholesterol.

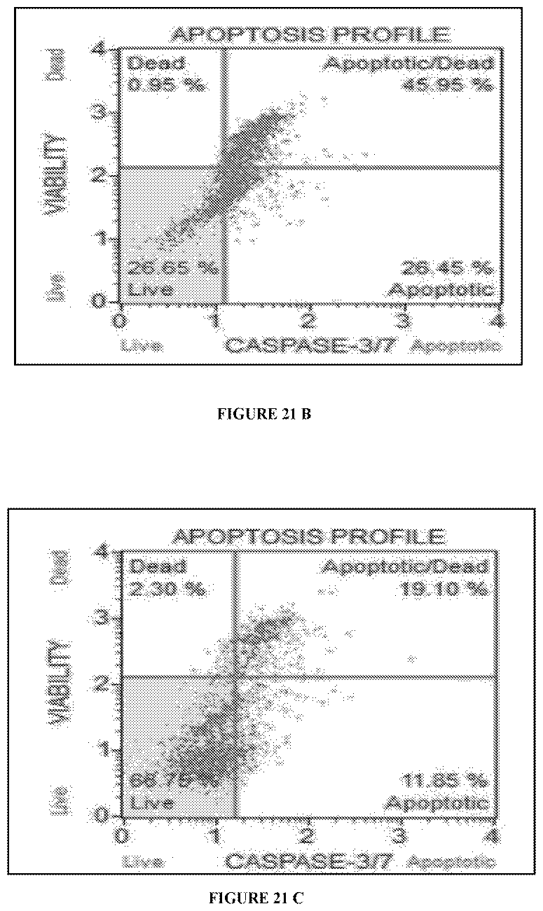

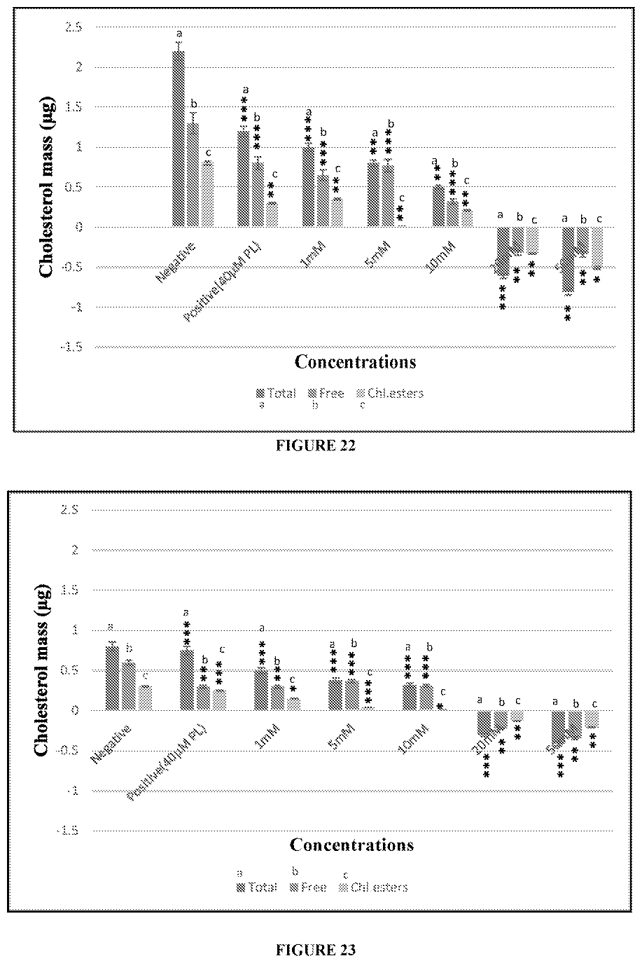

Results for MCF7 (FIG. 22) clearly showed that, as compared to untreated, the HPβCD treated cells had reduced total cholesterol content at all concentrations starting from 1 mM, 5 mM, 10 mM, 20 mM, and 50 mM. At 10 mM, which is the concentration we have screened for safe usage from our earlier assays, the total cholesterol levels were reduced to more than 75% as compared to the untreated sample.

MDA-MB-231 cells had lesser levels of cholesterol compared to MCF7 (FIG. 23). Results clearly showed that, as compared to untreated, the HPβCD treated cells had reduced total cholesterol content at all concentrations of HPβCD starting from 1 mM, 5 mM, 10 mM, 20 mM and 50 mM. At 10 mM the total cholesterol levels were reduced to more than 50% as compared to the untreated sample like MCF7 cells (FIG. 23).

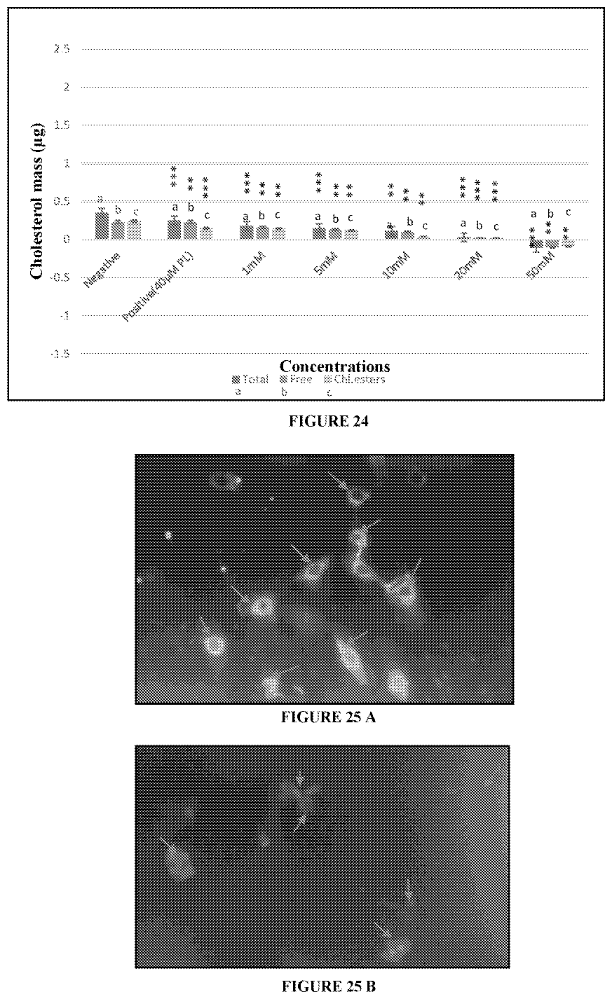

MRC-5 cells, being non-cancerous has lesser levels of cholesterol compared to both MCF7 and MDA-MB-231 cells because they divide less aggressively and therefore needs less cholesterol. Results clearly showed that, as compared to untreated, the HPβCD treated cells had reduced total cholesterol content at all concentrations starting from 1 mM, 5 mM, 10 mM, 20 mM, and 50 mM (FIG. 24). At 10 mM the total cholesterol levels were reduced to more than 50% as compared to the untreated sample similar to MCF7 and MDA-MB-231 cells but this depletion was enough to actually kill the cells as observed from our earlier assays. Upon quantifying the total cholesterol content it was seen that MCF7 and MDA-MB-231 cells has approximately 7.7 and 2.7 times more cholesterol as compared to the normal MRC-5 cells.

### Cholesterol Staining

To further validate cholesterol depletion as observed from the cholesterol assay above a Filipin based cholesterol staining was conducted. MCF7. MDA-MB-231 cells were used. HEK-293 cells could not be used due to unavailability. HPβCD and the positive (5 mM MBCD) were treated for 24 hrs. Like PL, MBCD is also a well know cholesterol depletor.

Fluorescence microscopy has been a robust tool for investigating the intracellular transport of proteins. Filipin specifically binds to cholesterol and can give an overall quantification and has been more recently been clinically used in the diagnosis of Type C Niemann-Pick disease.

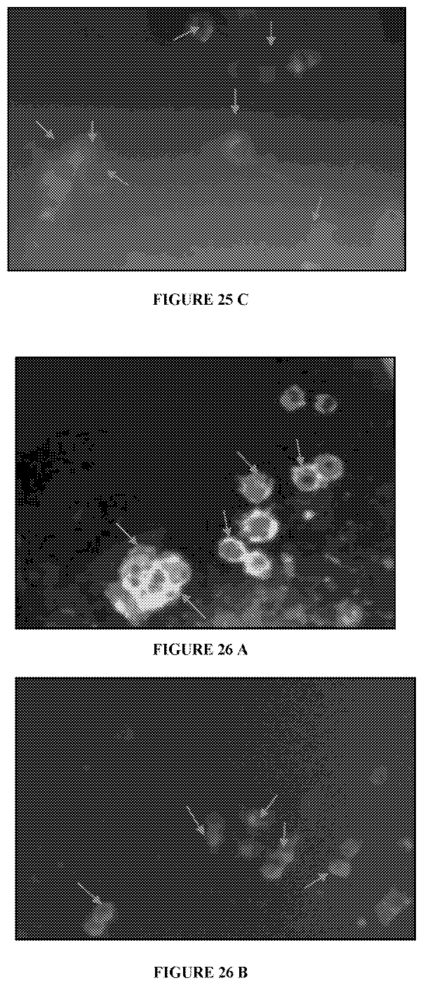

For MCF7 cells post-staining with Filipin, the untreated sample (FIG. 25 A) and the 10 mM HPβCD treated sample (FIG. 25 C) showed significant difference in terms of fluorescent intensity which was measured using NTH's ImageJ software. The treated cells had four times less fluorescence as compared to the untreated ones. This is in accordance to our cholesterol quantification assay where 10 mM HPβCD treatment caused almost 75% cholesterol depletion.

The positive (5 mM MBCD) had almost three times less fluorescence as compared to the untreated after quantification (FIG. 25 B).

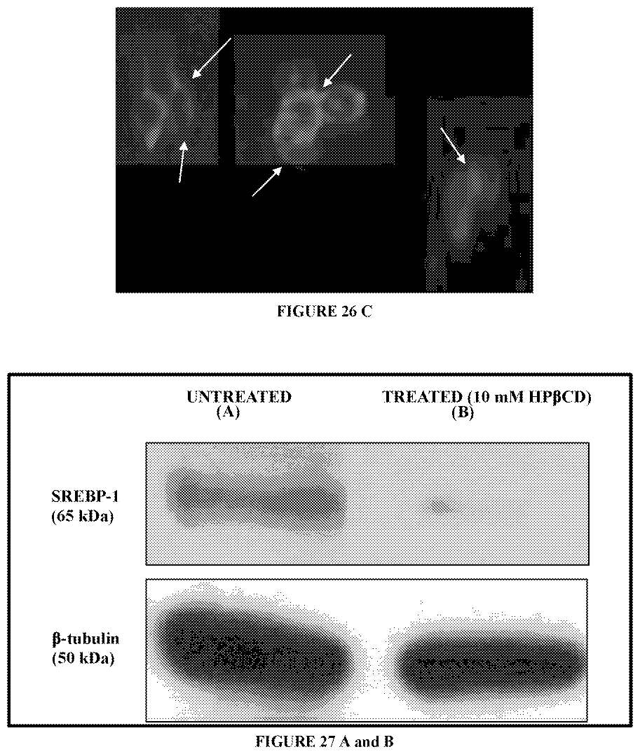

Similarly, for MDA-MB-231 cells there was a remarkable difference in the fluorescent intensity between the untreated (FIG. 26 A) and 10 mM HPβCD treated cells (FIG. 26 C).

After quantification with ImageJ, the treated cells had six times less fluorescence as compared to the untreated ones. This again, correlates with our data from cholesterol quantification assay where at 10 mM HPβCD, almost 50% total cholesterol w as seen to be depleted. The positive (5 mM MBCD) had almost eight times less fluorescence as compared to the untreated after quantification (FIG. 25 B). Thus, it can be affirmed that the apoptosis caused my HPβCD is indeed due to the overall depletion of cholesterol. Although studies could not be done on normal cells, but it is quite adequately known that they do have lesser cholesterol levels as compared to cancerous cells like mentioned before and therefore will show significantly lesser florescence.

### Western Blotting

In order to delve in to more detailed mechanisms of cholesterol depletion a western blotting analysis was conducted for SREBP-1 protein, which is a known transcription factor involved in cholesterol homeostasis. Lysates from MCF7, MDA-MB-231 and MRC-5 cells were used. 10 mM HPβCD was treated for 24 hrs. SREBPs are key elements responsible for the gene expression of vital enzymes involved in fatty acid synthesis which includes SREBP 1 and SREBP 2. Down regulation of SREBPs potentially correlates to regression in cell growth and migration and has also been shown to cause apoptosis in several cancers. In order to investigate this characteristic feature of SREBPs a western blot analysis was conducted to see if HPβCD treatment does alter SREBP levels.

For MCF7s, after quantification using the Image Lab™πSoftware, it was seen that the treated (10 mM HPβCD) protein sample (FIG. 27 A) had about five times less activity of SREBP-1 as compared to the untreated lysate (FIG. 27 B) after equal loading of the respective samples. Thus, indicating that SREBP-1 is significantly downregulated upon HPβCD treatment.

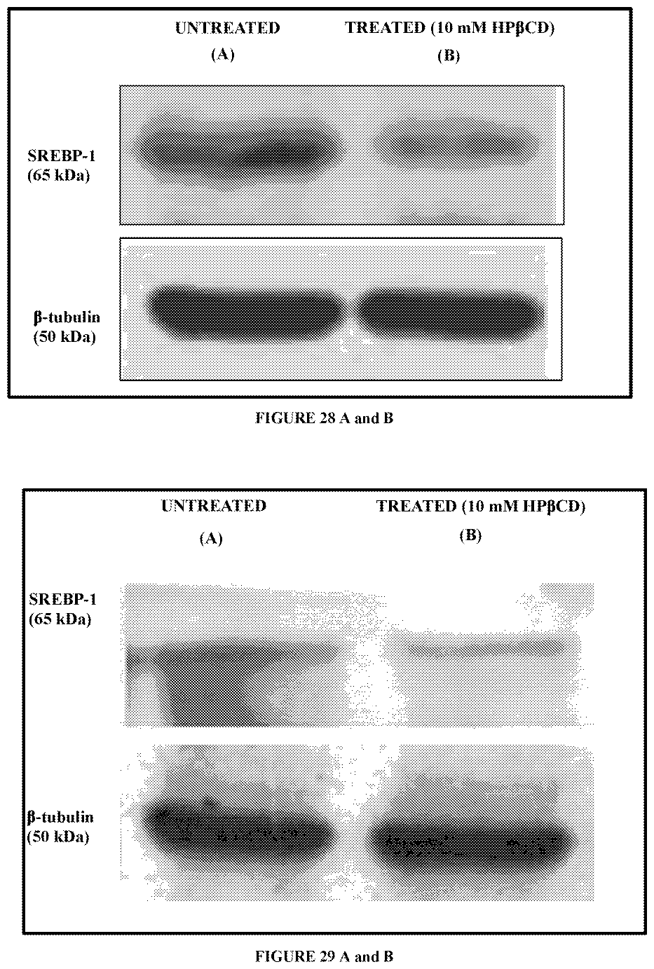

In MDA-MB-231, a similar observation was made. Post quantification using the Image Lab™ Software, it was seen that the treated (10 mM HPβCD) protein sample (FIG. 28 A) had about two and a half times less activity of SREBP-1 as compared to the untreated lysate (FIG. 28 B) after equal loading of the respective samples.

Interestingly in the normal MRC-5 cells, after quantification it was observed that SREBP-1 activity remained equal and stayed at basal levels both in the untreated (FIG. 29 A) and treated lysates (FIG. 29 B). This western blotting analysis is a confirmation and proves that HPβCD treatment knocks down SREBP-1 activity in cancer cells remarkably whereas in normal cells it stays the same and does not impact on cholesterol biosynthesis

### In Vivo Studies (Mice Xenografts)

Since the potency of HPβCD against breast cancer was established in our in vitro assays, a thorough investigation in a mice model was conducted (Nude Mice. MF-1 strain). MCF-7 and MDA-MB-231 cells were injected in 32 mice (16 for each cell type) and after tumour development, was treated with HPβCD (3000 mg/kg b.w.). Thrice a week dose treatment plan was followed throughout the study.

To examine if HPβCD can be used as a future anti-breast cancer drug, mice xenografts were prepared. MCF7 cells (5 million) cells were injected in 16 mice with weekly β-estradiol supplementation. The study for triple negative induced breast cancer (MDA-MB-231) is complete. Up to 4 million cells were injected in 16 mice. This was subdivided in three more groups namely: Late stage, intermediate stage and early stage estrogen negative cancer. 4 mice were grouped as untreated (not receiving HPβCD), 4 for treated (late stage), 3 for treated (intermediate stage) and 3 for treated (early stage).

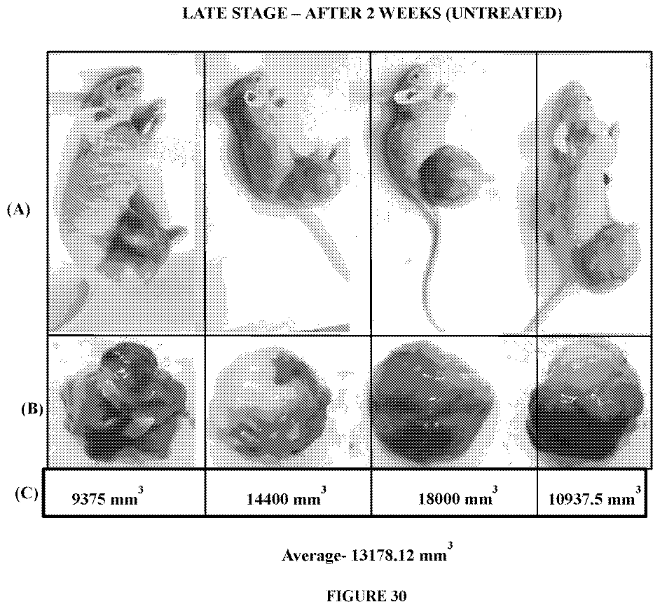

After about 5 weeks post cells injection, tumours were observed in the untreated group (n=4) and the average size went up to 13178.12 mm³ in two weeks' time (FIG. 30 A, B, C). This is because MDA-MB-231 cells are extremely aggressive in nature and that is the reason there is any adequate treatments except the side effects prone to chemotherapy. These 4 mice had to be euthanized at this point because of humane end points. In the late stage treated group, the treatment commenced when the size of the tumour was in between 900 mm³ to 1350 mm³ (FIG. 30 A, B, C). After following a strict 10 dose approach where HPβCD was administered intraperitoneally 10 times (once each day) for a period of 2-3 weeks, we saw significant reduction in the progression of tumours and the average size of the tumours reduced to a significant 73.9% (FIG. 30 C) compared to the untreated group.

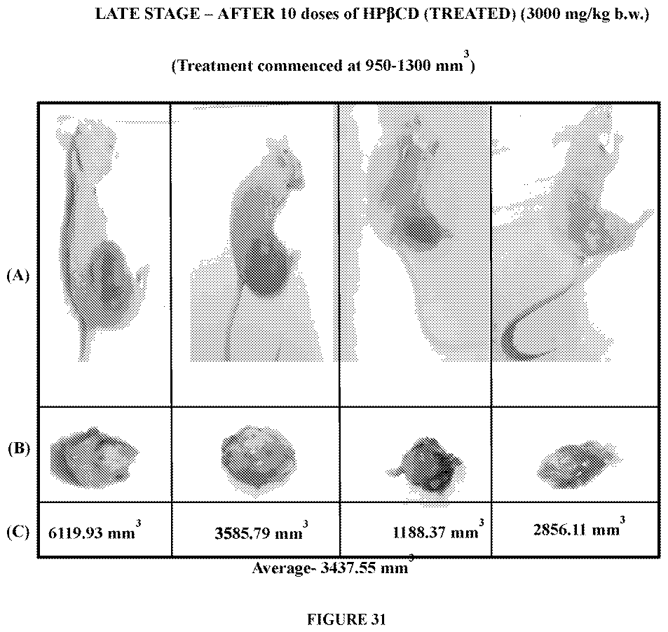

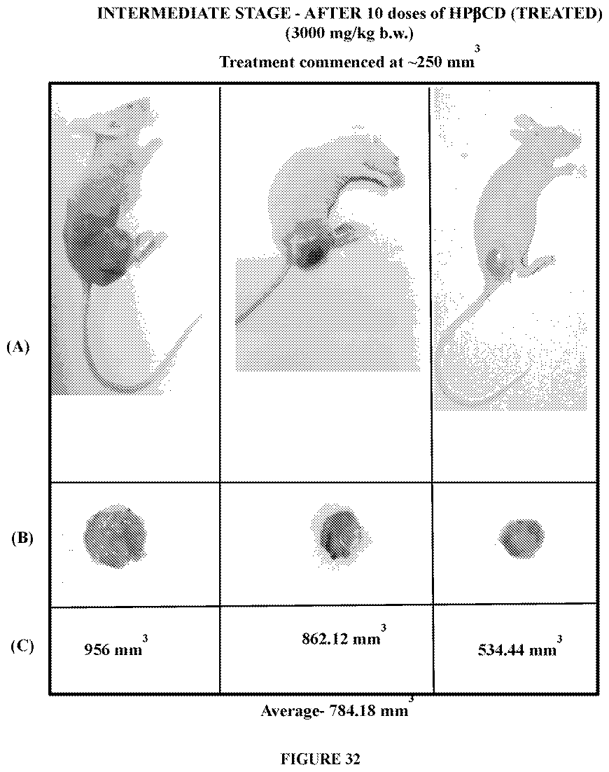

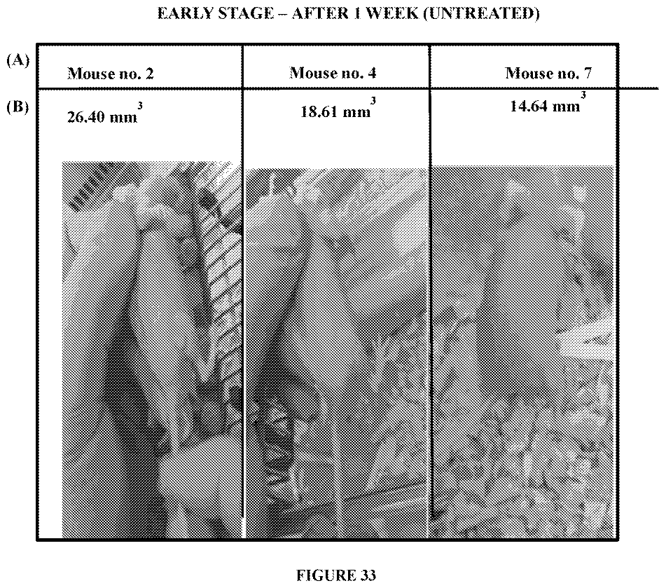

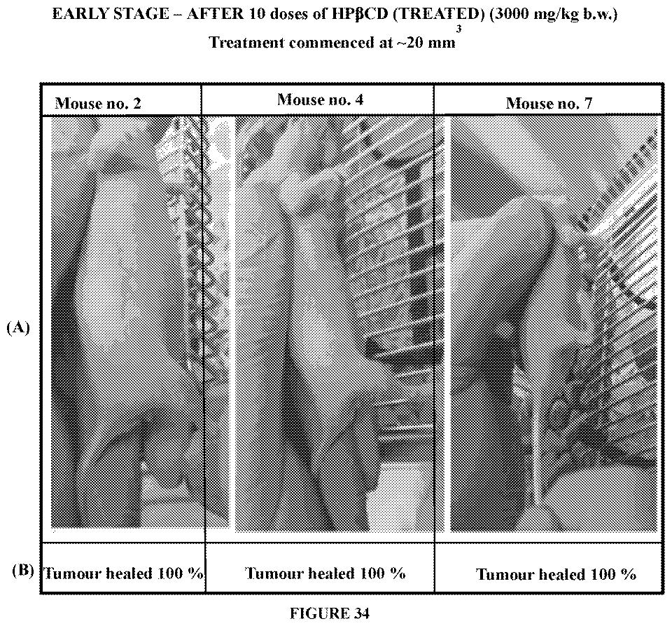

In the intermediate stage group (n=3), the treatment commenced when the size of the tumours reached ˜250 mm³. Again, after 10 doses of HPβCD treatment we saw the average size of the tumours went down to 784.18 mm3 which is a staggering 94% decrease (FIG. 31 B, C) as compared to the untreated group. This was remarkable considering how invasive MDA-MB-231 cells are and HPβCD's ability to tame them was quite phenomenal. Finally, in the early stage group (n=3), the treatment commenced when the tumour size was 20 mm³ (FIG. 33 A. B). HPβCD was administered for 10 dosages and after about a period of one and a half weeks, the tumours were completely healed (FIG. 34 A, B). These 3 mice were then kept for a period of 4 weeks to check for relapse of tumour because estrogen negative tumours have an extreme propensity to recur.

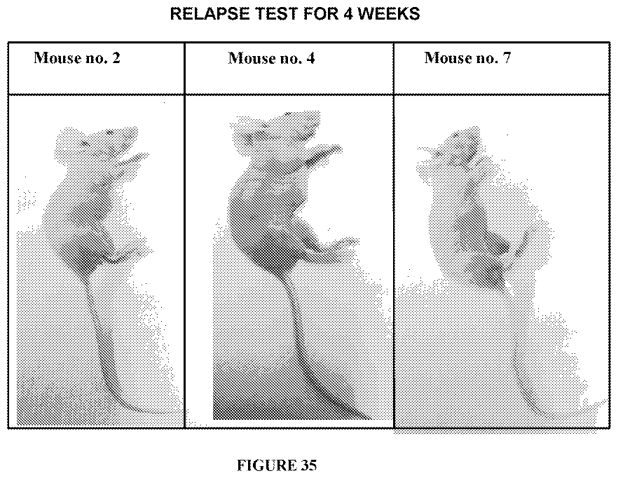

After 4 weeks of the relapse test (FIG. 35) it was clearly observed that there was no recurrence of the tumour. These mice could not be kept for longer periods due to financial and technical constraints.

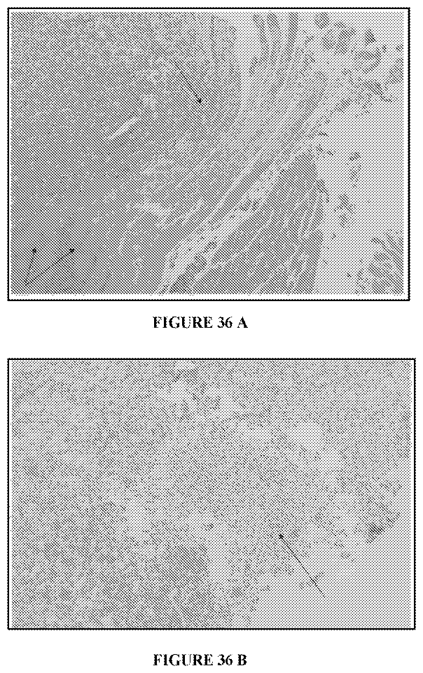

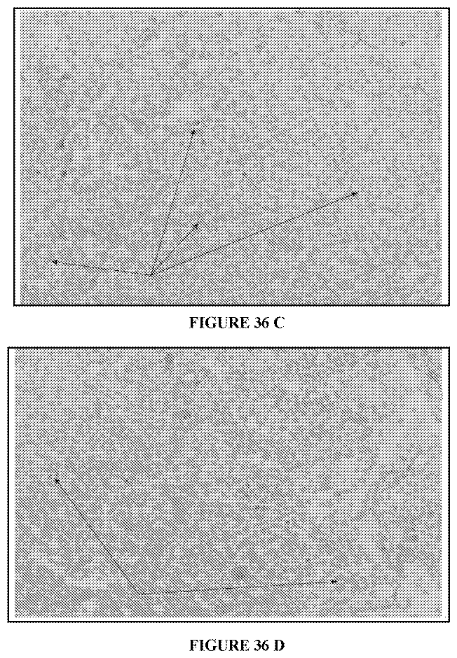

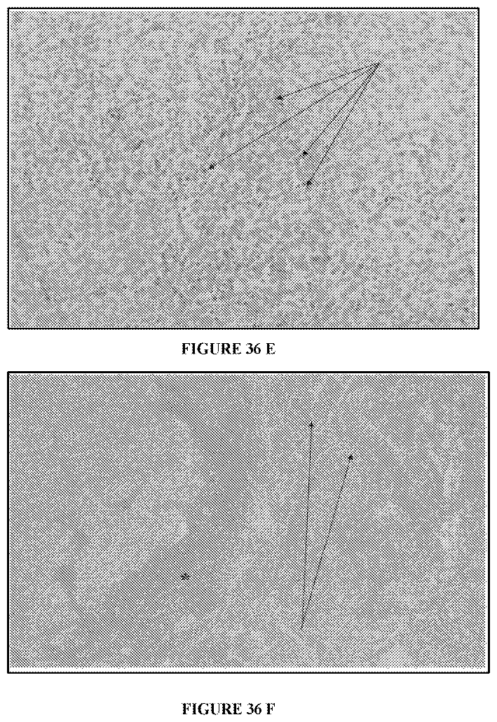

Hematoxylin and cosin staining of mice was undertaken in untreated, intermediate and late stage MDA-MB-231 mice as is shown in FIG. 36.

### Conclusions (In Vivo Studies)

The Applicant believes that 2-hydroxypropyl-β-cyclodextrin (HPβCD) for use in the treatment and/or prevention of breast cancer, particularly triple negative breast cancer, at least ameliorates the disadvantages known in the prior art where it is typically known that triple negative breast cancer remains unresponsive to chemotherapeutic treatment protocols. Moreover, use of HPβCD includes none of the severe side effects of typical chemotherapeutic treatment protocols, therein improving patient compliance and quality of life for the human or animal undergoing treatment of breast cancer.

### Hematoxylin and Cosin Staining of Mice:

Microscopic examination of the tumours (see Table 2 and FIG. 36) shows a solid growth pattern with some sections demonstrating neoplastic cells dissecting between skeletal muscles. Hematoxylin and eosin examination of untreated, intermediate and late in MDA-MB-2-3 mice is shown in FIG. 36. The individual cells are markedly pleomorphic and have raised nuclear to cytoplasmic ratios. The neoplastic cells have irregular nuclear borders and the cells have vesicular chromatic with one to multiple prominent nucleoli. Brisk mitotic activity is evident. Areas of perineural and lymphovascular invasion are however, not documented.

Sections of the tumour in the untreated group show focal areas of necrosis at the peripheries accounting for 1-5% of the tissue examined. One case (UT3) demonstrates extensive muscle invasion. Apoptotic cells are identified. One case (UT 10) from this group of tumours demonstrates the greatest number of mitoses (77 per 10 high powered fields). Numerous atypical mitoses are present. Sections from the intermediate group of tumours show scattered foci of necrosis throughout the tumour accounting for approximately 6-10% of the total tumour examined. Brisk mitotic activity is noted, similar to that seen in the untreated group. Atypical mitotic figures are observed. The number of apoptotic cells in this group of tumours is comparable to that seen in the untreated group.

Sections from the late group show pockets of necrosis throughout the tumour with some demonstrating large, geographic regions of necrosis. The areas of necrosis are far more prominent than that visualised in the other two groups. One case (Late 12) shows necrosis accounting for up to 20% of the tissue examined, thus illustrating approximately a 4-fold increase in necrosis compared to that noted in the untreated group. Greater numbers of apoptotic cells are noted in this group of tumours with the late group showing approximately a 1.7-fold increase in the number of apoptotic cells compared to the untreated group and 2.9 times more apoptotic cells than the intermediate group. Mitotic activity in the late group is approximately 1.1 times lower than that noted in the other two groups of tumours.

TABLE 2
Case study of MDA-MB-231 hematoxylin and eosin staining (H&E) sections

| | | EPID/ | APOPTOSIS/ | LV1/ | |
|---|---|---|---|---|---|
| CASE | NECROSIS | MUSCLE INV. | 10 HPF | PN1 | MITOSES |
| UT1 | Present at | Muscle invasion No | 17 | — | 59 |
| | periphery | epidermis | | | |
| UT3 | Focal at the | Extensive muscle | 22 | — | 65 Numerous |
| | periphery | invasion | | | atypical forms |
| UT9 | Present at the | Extensive muscle | 27 | — | 57 Atypical |
| | periphery | invasion. No epidermis | | | forms present |
| INTM | Focal necrosis | Muscle infiltration at | 10 | — | 70 Atypical |
| 13 | | the periphery | | | forms present |
| INTM | Occasional | No muscle or epidermis | 21 | — | 59 Atypical |
| 31 | pockets of | | | | forms present |
| | necrosis | | | | |
| INTM | Multiple pockets | Muscle invasion. No | 23 | — | 67 Atypical |
| 32 | of necrosis | epidermis | | | forms present |
| LATE | Scattered foci of | No muscle present | 21 | — | 60 |
| 8 (1) | necrosis | | | | |
| LATE | Scattered foci of | No muscle or epidermis | 30 | — | 65 |
| 8 (2) | necrosis | | | | |
| UT 10 | Scattered areas of | Muscle invasion | 22 | — | 77 |
| | necrosis | | | | |
| LATE | Scattered foci of | Muscle invasion. | 39 | — | 57 |
| 11 | necrosis present | Minimal uninvolved | | | |
| | at the periphery | epidermis | | | |
| LATE | Necrosis seen | Muscle invasion at | 32 | — | 59 |
| 12 | centrally and | periphery. No | | | |
| | peripherally | epidermis present | | | |
| LATE | Pockets of | Muscle invasion. | 27 | — | 50 |
| 14 | necrosis scattered | Epidermis is | | | |
| | throughout | uninvolved | | | |

Therefore, in conclusion from the mice studies it can be inferred that possibly in humans, if the tumour is diagnosed at a late stage or intermediate stage, HPβCD treatment might slow the progression of the cancer and thereby improve human life expectancy and if it detected at an early stage. It is also postulated that it may be possible to cure the breast cancer, typically triple negative breast cancer, completely without any severe complexities and side-effects of chemotherapy.

HPβCD does not Induce Liver Toxicity

### AST/ALT Assay

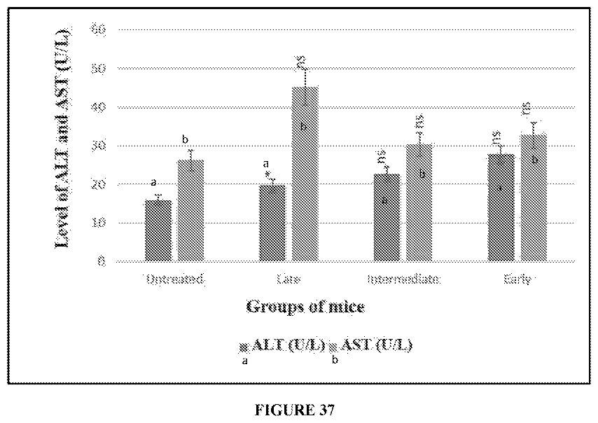

Post-euthanisation, the mice serum was analysed for AST/ALT (alanine aminotransferase/aspartate aminotransferase) levels in order to observe if HPβCD treatment had any toxic effects on liver of treated mice (FIG. 37). The AST/ALT ratio gives an estimate of liver toxicity or damage. The tests revealed that HPβCD did not have toxic effects on the mice. The AST/ALT ratio for all the early and intermediate groups was not different from the untreated group, was under the recommended ratio of 2 and the values were below the reference range (40 U/L) except for AST levels in the late group. In latter groups, the ratio of AST/ALT was 2.2 which was slightly above the recommended limit but was not found to be statistically different from the untreated group. These results show that HPβCD caused no toxicity when administered to the mice. In this regard review FIG. 37.

On MDA-MB-231 cells further work was carried out to identify the mechanism of action in cancer cell model (using RT-PCR [reverse transcriptase polymerase chain reaction] arrays and staining), and to validate the mechanism of action and confirm drug-protein interactions as identified above.

### Identifying Mechanism of Action of HPβCD

PCR (polymerase chain reaction) arrays were run to identify gene expression changes in cancer cells after treatment with HPβCD. The differentially expressed genes along with their significance for cancer, some genes were selected for further drug-protein interaction studies.

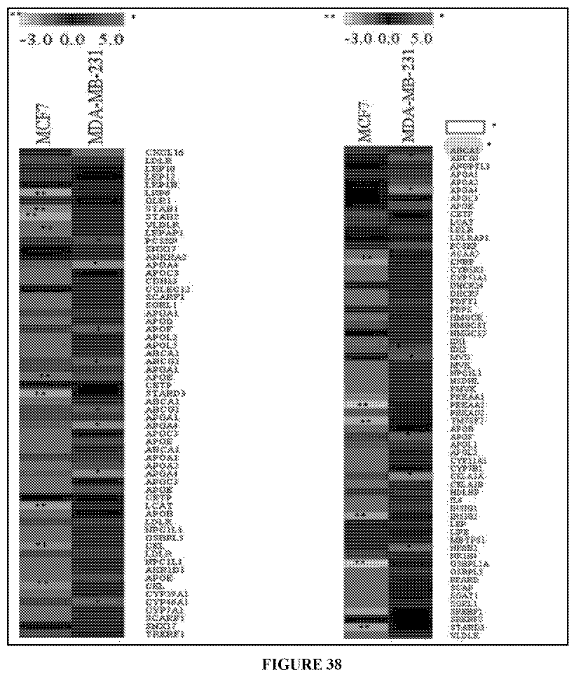

Results from RT² Profiler™ PCR Array Human Lipoprotein Signaling & Cholesterol Metabolism (MCF7 and MDA-MB-231) are shown in FIG. 38.

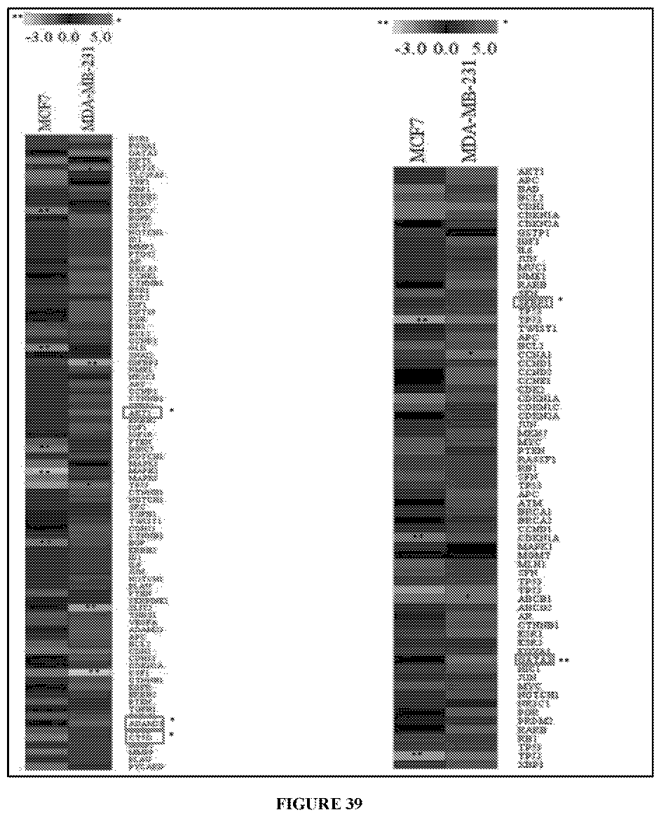

Results from RT² Profiler™ PCR Array Human Breast cancer (MCF and MDA-MB-231) are shown in FIG. 39.

Five genes were selected from this array post gene expression analysis. In MDA-MB-231 cells, it was observed that Akt1 was upregulated which suggests decrease in migration of breast cancer cells, for instance by regulating TSC2, palladin and EMT-proteins. SFRP1, which is an important inhibitor of the Wnt pathway and is a known tumour suppressor gene, which is epigenetically silenced in a variety of tumours was also seen to be upregulated. Interestingly, GATA3, which is a useful marker for luminal category tumours particularly breast cancer was downregulated upon treatment with HPβCD suggesting the efficacy of the compound as a possible treatment. Previous studies have highlighted that CTSD gene transcription is increased by estrogen and growth factors in estrogen-receptor-positive breast cancer cells and by an unknown mechanism in estrogen-receptor-negative cells thus making it an interesting candidate to investigate. From the above gene expression investigation it was observed that CTSD was upregulated in MDA-MB-231 cells whereas it was significantly downregulated in MCF7 which may suggest that treatment with HPβCD is mostly likely to be the causative agent. Last but not the least ADAM23 was selected which causes breast cancer progression and dissemination of mesenchymal circulating tumour cells. In our case, ADAM23 was upregulated in MDA-MB-231 cells and downregulated by many folds in MCF7 which makes it an interesting target to investigate and can in all respects, validate HPβCD's efficacy as a potential dg and also to investigate underlying mechanism of action of HPβCD.

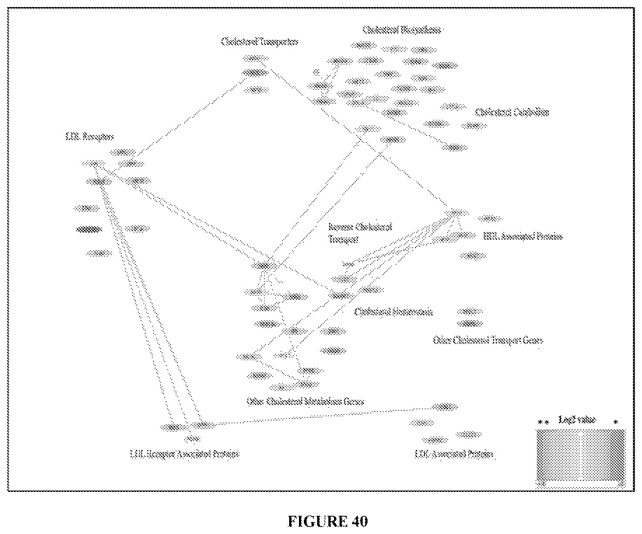

The representation in FIG. 40 shows that after treatment with HPβCD, majority of genes involved in cholesterol related pathways were downregulated. This information has been generated for the first time in cancer cells. This observation strongly suggests that HPβCD acts by disturbing cholesterol homeostasis in cancer cells and cholesterol depletion can be used as a therapeutic strategy to treat cancer.

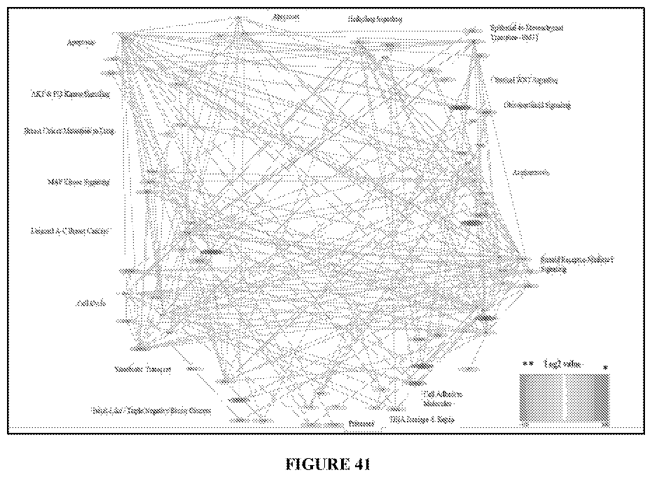

These results demonstrate that HPβCD blocks several signalling pathways in the cancer cell especially DNA damage and repair, AKT signalling, Hedgehog signalling and EMT (epithelial to mesenchymal transition) which is responsible for metastasis of tumour. No significant change was observed in steroid-receptor mediated signalling, thus suggesting that HPβCD is not dependent on hormonal receptors to induce its action. (see FIG. 41).

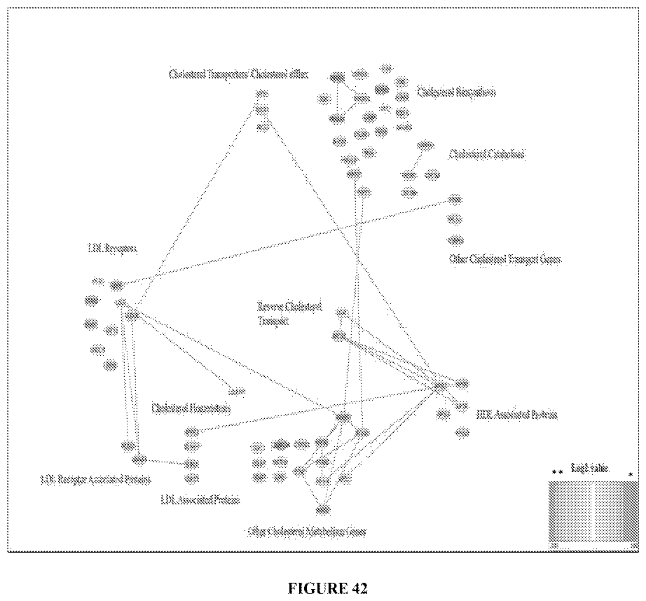

The representation in FIG. 42 shows that after treatment with HPβCD, majority of genes involved in cholesterol related pathways were downregulated similar to MDA-MB-231 cells. This observation strongly suggests that HPβCD acts by disturbing cholesterol homeostasis in cancer cells and cholesterol depletion can be used as a therapeutic strategy to treat cancer.

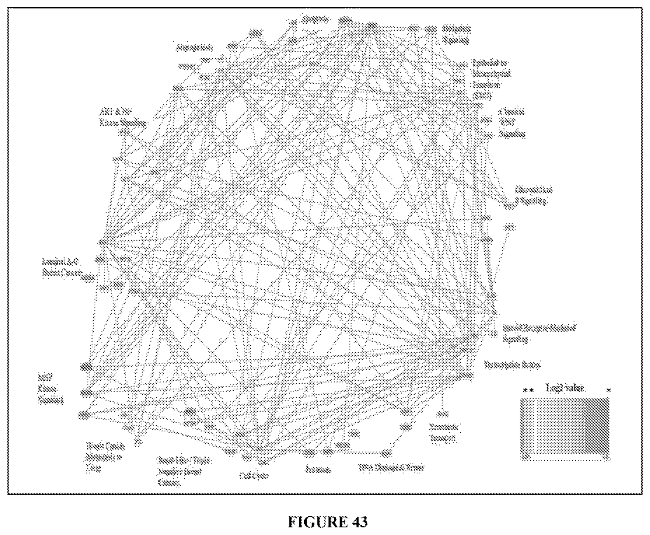

The above results demonstrate similar trends like obtained for MDA-MB-231 cells and it was observed that HPβCD blocks signalling pathways like the DNA damage and repair. AKT signalling. Hedgehog signalling and EMT (epithelial to mesenchymal transition) which is responsible for metastasis of tumour. Again, no significant change was observed in steroid-receptor mediated signalling, thus suggesting that HPβCD is not dependent on hormonal receptors to induce its action. (see FIG. 43).

### Confirming Mechanism of Action

### Identifying Drug-Protein Interactions Between HPβCD and Selected Proteins Based on RT-PCR Array Data

Six proteins were selected from the arrays: Akt1, ADAM23, Cathepsin D (CTSD), SFRP1, GATA3 and ABCA1 based on their gene expression analysis in both MCF7 and MDA-MB231, with major focus on MDA-MB-231 because mice studies were completed with the same.

### Determination of Binding Affinity for HPβCD for Akt1, ADAM23, Cathepsin D (CTSD), SFRP1, GATA3 and ABCA1.

The binding affinity for HPβCD for the six human proteins assay was conducted using the BioNavis™ 420A ILVES MP-SPR (BioNavis, Tampere, Finland) at 25° C. As running buffer, degassed PBS Tween 20 was used. The recombinant proteins were immobilised as ligands at 0.5 ug/ml onto functionalized 3D carboxymethyl dextran sensors (CMD 3D 500L; BioNavis, Tampere. Finland). Immobilization of ligands was achieved through amine coupling after 1-Ethyl-3-(3-dimethylaminopropyl) carbodiimide (EDC) [Sigma Aldrich, Germany] and N-Hydroxy-succinimide (NHS) [Sigma Aldrich, Germany] activation following a protocol provided by the manufacturer (BioNavis. Finland) to achieve <200 RUs. A reference channel without immobilized protein served as control for non-specific binding and changes in refractive index. As analytes HPβCD was prepared into aliquots of 0, 1.25, 2.5, 5 and 10 nM injected three times at a flow rate of 50 μl/min into each flow cell. Injections with buffer only were used as controls. Association between ligand and analyte was allowed for 3 min and dissociation was monitored for a total of 10 min. Kinetics steady-state equilibrium constant data was processed after double referencing of the sensograms using global fitting using TraceDrawer software version 1.8 (Ridgeview Instruments, Sweden).

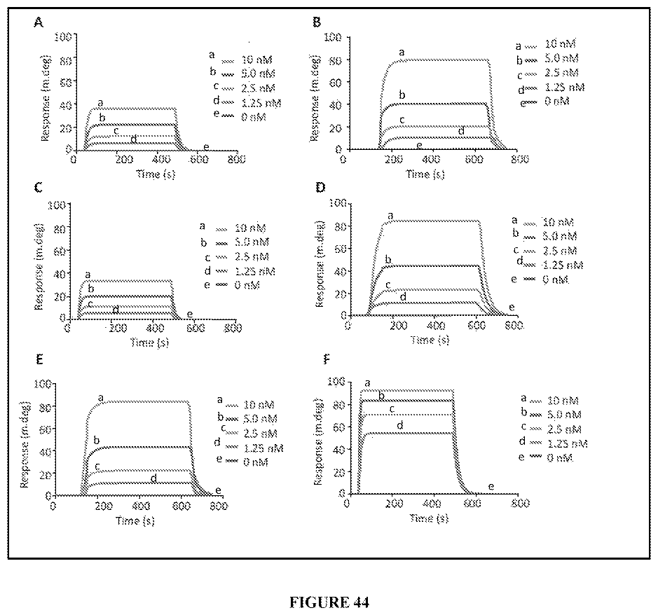

HPβCD interacts with the six human proteins (FIG. 44). The kinetics analysis of HPβCD with the human proteins showed the highest affinity with SFRP1 in the nanomolar range and the ADAM3 protein had the lowest binding affinity in the micromolar range (Table 3).

TABLE 3
Kinetics data for the interaction of HPCD with human recombinant proteins

| Ligand | Kₐ (1/(M*s)) ± est. error | K_{d} (1/s) ± est. error | K_{D} (M) ± est. error | X² |
|---|---|---|---|---|
| ABCA1 | 3.55 (±0.003) e⁺⁰⁶ | 6.27 (±0.012) e⁻⁰² | 1.77 (±0.46) e⁻⁰⁸ | 1.45 |
| ADAM3 | 3.73 (±0.27) e⁺⁰⁵ | 7.58 (±0.32)e⁻⁰² | 2.03 (±0.15) e⁻⁰⁷ | 7.19 |
| AKT1 | 3.98 (±0.64) e⁺⁰⁶ | 7.45 (±0.11) e⁻⁰² | 1.87 (±0.57) e⁻⁰⁸ | 2.41 |
| GATA3 | 6.57 (±0.39) e⁺⁰⁵ | 5.92 (±0.36) e⁻⁰² | 9.00 (±0.59) e⁻⁰⁸ | 1.09 |
| Cathepsin | 4.40 (±0.14) e⁺⁰⁵ | 5.63 (±0.14) e⁻⁰² | 1.28 (±0.44) e⁻⁰⁷ | 4.0 |
| (CTSD) | | | | |
| SFRP1 | 1.03 (±0.51) e⁺⁰⁷ | 8.12 (±0.34) e⁻⁰² | 7.91 (±0.72) e⁻⁰⁹ | 2.23 |

The direct interaction kinetics of HPβCD with various human proteins is represented by the rate of association (Ka), rate of dissociation (Kd) and the steady state affinity (K_{D}) as determined by SPR analysis. The ligand represents the respective immobilised protein on the CMD 3D 500L chip surface to which the analyte (HPβCD) was injected at a flow rate of 50 μl/min for at least three times. Data were analysed after double referencing (refractive index changes on chip surface without protein immobilisation and for buffer injected without analyte) as baseline. Data are represented as mean plus/minus standard error of measurement. Chi square (χ²) values determined show the 1:1 langmuir curve fitting residuals.

### CONCLUSION

1. The affinity for HPβCD to bind the proteins are as this order, high affinity: SPRF1>ABCA1>AKT1>GATA3>Cathepsin>ADAM3, low affinity.

2. Rate of association, fast binding: SFRP1>AKT1>ABCA1>GATA3>Cathepsin>ADAM3 slowest binding.

3. Rate of dissociation, fast to dissociate: SRFP1>ADAM>AKT>ABCA>GATA3>Cathepsin slow to dissociate.

Cholesterol Staining of Tumour Tissue from Mice Using Filipin

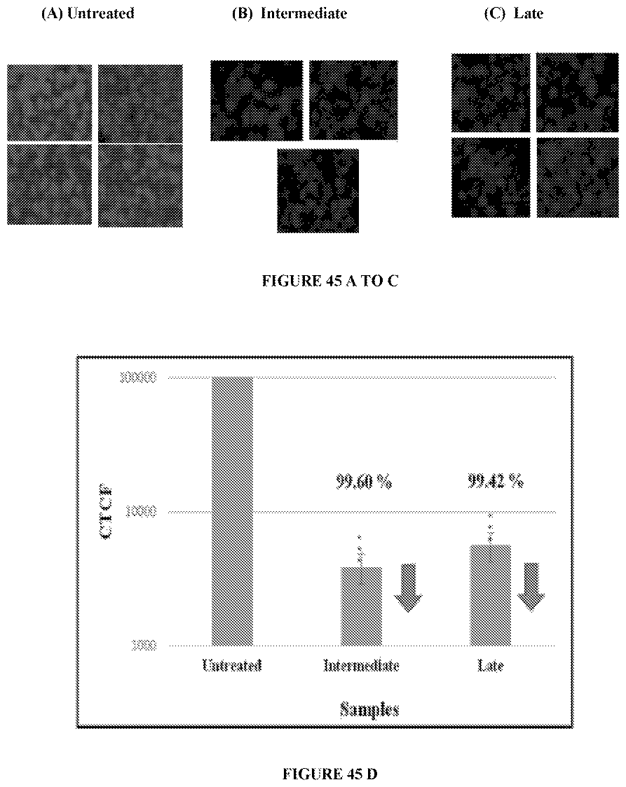

FIG. 45 shows Filipin staining (for cholesterol) of mice sections. 4 from untreated, 3 from intermediate and 4 from late stage. Results show decreased cholesterol in intermediate and late stage tumours compared to untreated.

The results show that HPβCD reached tumours and extracted cholesterol from tumours as significant reduction in cholesterol was identified. This confirms that HPβCD reduces tumour size by extracting cholesterol, which stops proliferation of cancer cells.

### Cholesterol Staining of Cancer Cells Using Filipin

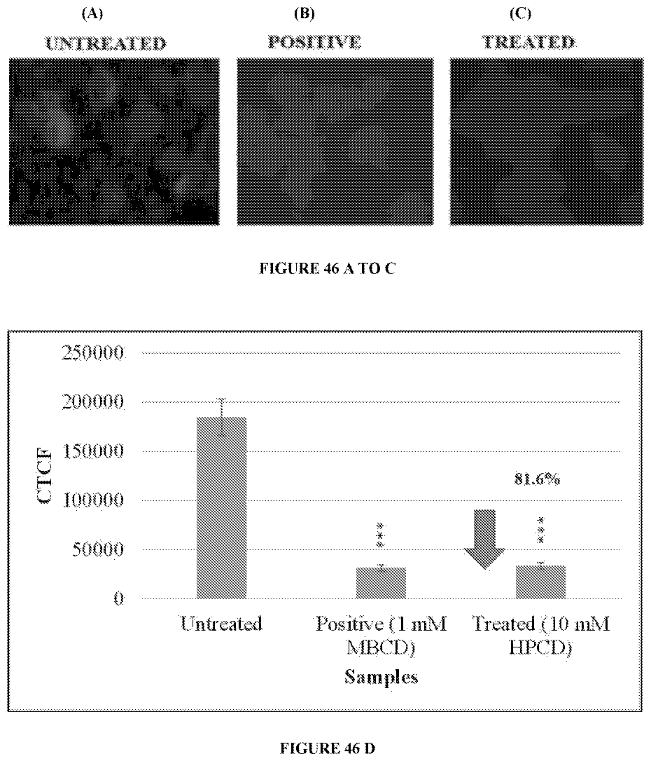

FIG. 46 shows Filipin staining of MDA-MB-231 cells for cholesterol. Results show decreased cholesterol in treated cells compared to untreated.

### Lipid Staining of Cancer Cells Using BODIPY

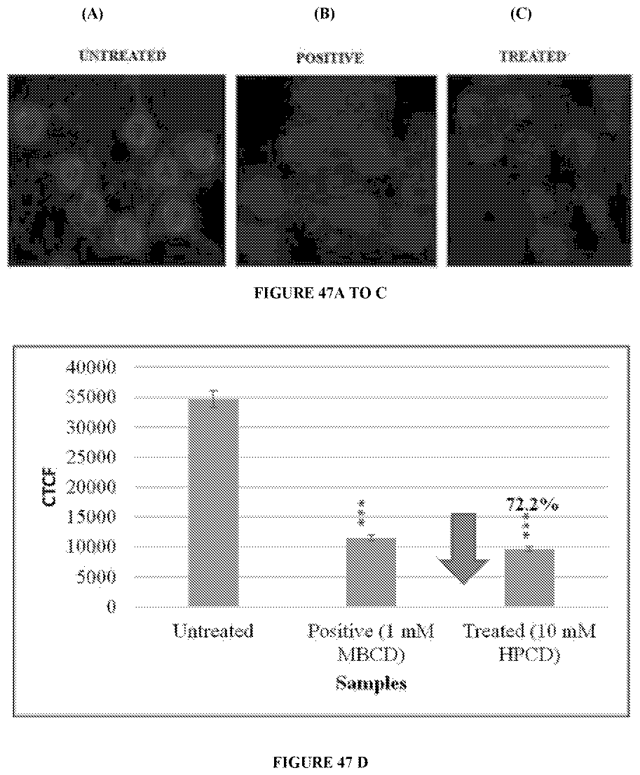

FIG. 47 shows BODIPY staining of MDA-MB-231 cells for lipid droplets. Green shows lipid staining while blue is the nuclear stain. Results show decrease in lipid droplets in treated cells compared to untreated. Lipid droplets store cholesterol and HPβCD was able to reduce accumulation and storage of cholesterol in cancer cells.

Staining of Lipid Rafts in Cancer Cells using Alexa Fluor

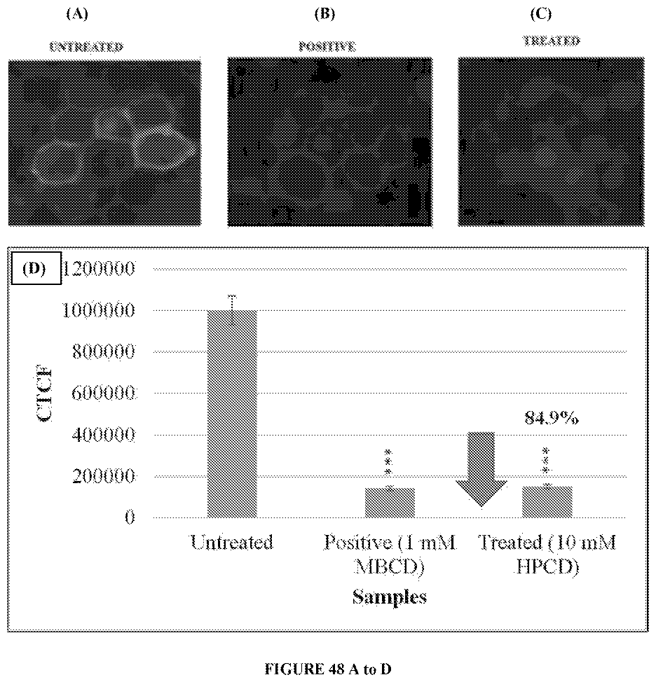

FIG. 48 shows ALEXA FLUOR staining of MDA-MB-231 cells for lipid raft dissociation. Results show decrease in lipid rafts in treated cells compared to untreated.

### CONCLUSIONS

Different activities carried out have allowed us to establish:

1. The anti-tumour potential of HPβCD in cell and mice models.

2. Identify the mechanism of action in cancer cell model, and

3. Validate the mechanism of action and confirm drug-protein interactions.

We have confirmed that HPβCD impacts several molecular mechanisms in the cancer cell, especially cholesterol related pathways (as confirmed in mice tumour samples and RT-PCR data), thus establishing a proof for HPβCD's anticancer potential. We also identified several protein targets in the cell that have been confirmed to bind with HPβCD. One of the key protein was SFRP1, which has been known to regulate cancer related signalling pathways, that has shown strongest binding with HPβCD in nanomolar range. It means that HPβCD stabilizes the protein for its action in the cell. Down regulation of SFRP1 has been found in many cancers including breast cancer, therefore, prolonged expression of this protein would help to prevent cancer progression. In our studies, we have found that HPβCD upregulates expression of mRNA of SFRP1 and it also binds to the protein.

In conclusion, we have confirmed that HPβCD inhibits tumour growth and progression by depleting cholesterol from cancer cells which is the result of molecular level changes in several cholesterol related pathways. The effects are amplified by modulation of several cancer related signalling pathways, ultimately inhibiting cancer cell growth. The main advantage of HPβCD is safety as it is not toxic to humans (as confirmed by previous studies), thus offers a totally new avenue for cancer therapeutics.

We have shown the efficacy of HPβCD against breast cancer tumours especially in triple negative breast cancer (TNBC). We have also elucidated its mechanism of action of the molecule. HPβCD can be an extremely cost-effective treatment for patients suffering from TNBC, which till date has no treatment other than surgery and chemotherapy both of which has severe side effects, and survival rates are less than 5-years.

### Further Work was Also Carried Out on MCF-7 Cells Induced ER+ Breast Cancer Mice Model.

### HPβCD Significantly Reduces Tumour Size in Estrogen Positive Breast Cancer

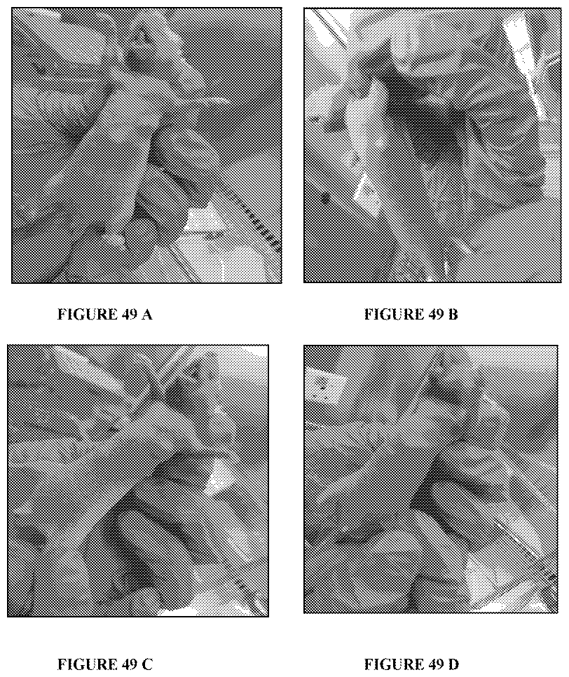

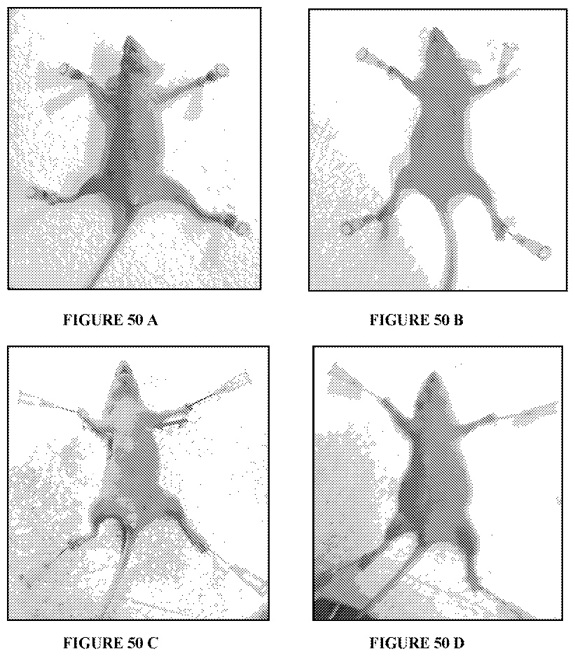

The anticancer effect of HPβCD on estrogen positive MCF7 cells induced tumours in xenograft mice model was also tested. The study was not completed as only 4 out of 16 mice developed tumours. But the mice who developed tumour were treated with HPβCD and 9.6% tumour reduction was observed (FIGS. 49 and 50).

FIG. 49 show photographs which shows development of tumours in 4 mice (ER+). (A) shows Mouse No. 29 tumour 173.21 mm³, (B) shows Mouse No. 30 tumour 163.46 mm³, (C) shows Mouse No. 31 tumour 188.01 mm³, and (D) shows Mouse No. 32 tumour 103.74 mm³.

FIG. 50 (A) to (D) shows photographs of ER+ Mice treated with HPβCD for 5 weeks. (A) shows Mouse no. 29 wherein the tumour healed completely. (B) shows Mouse No. 32 wherein the tumour healed completely, (C) shows Mouse No. 31 wherein the tumour reduced to 21.28 mm³, and (D) shows Mouse No. 30 wherein the tumour healed completely. Avery tumour size 5.3 mm³. Tumour reduction 96.6%.

### Cholesterol Staining of Cancer Cells Using Filipin

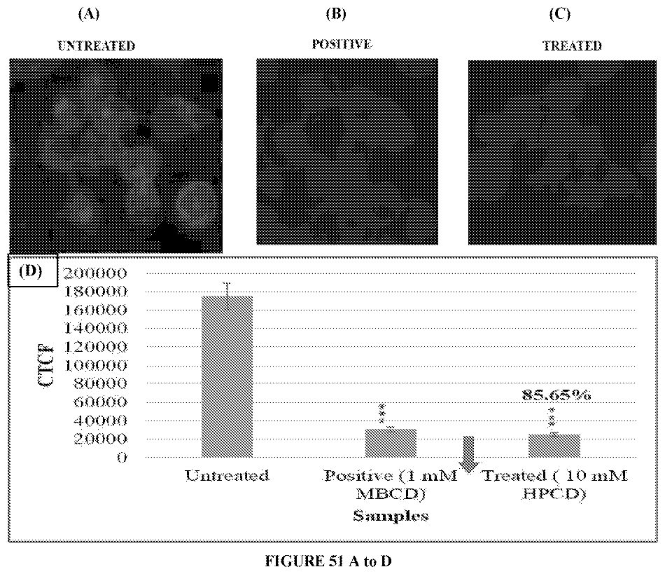

Results show decreased cholesterol in treated cells compared to untreated (see FIG. 51). FIG. 51 (A) to (C) shows Filipin staining of MCF7 cells for cholesterol, wherein (A) is untreated, (B) is positive and (C) is treated. (D) graphically shows decreased cholesterol in treated cells when compared to untreated cells.

### Lipid Staining of Cancer Cells Using BODIPY

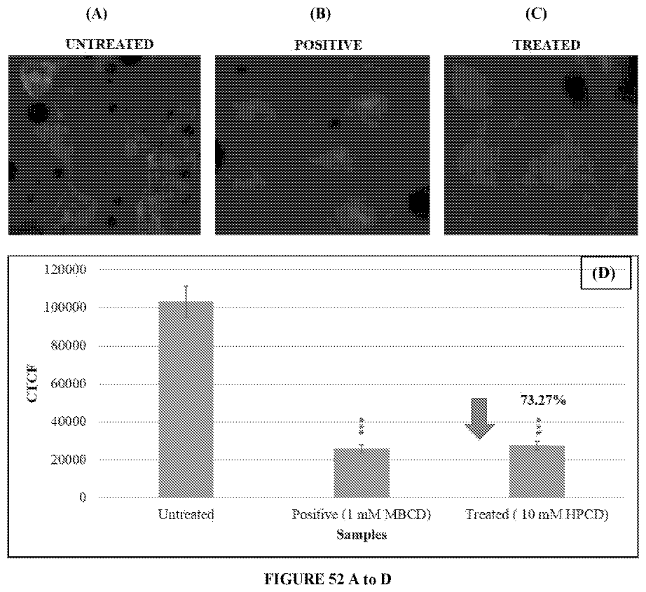

Results show decrease in lipid droplets in treated cells compared to untreated. Lipid droplets store cholesterol and HPβCD was able to reduce accumulation and storage of cholesterol in cancer cells (see FIG. 52). FIG. 52(A) to (C) shows BODIPY staining of MCF7 cells for lipid droplets. Green (darker areas in black and white figures) shows lipid staining while blue (lighter defined areas in black and white figures) is the nuclear stain. (A) is untreated, (B) is positive and (C) is treated. (D) shows graphically the results wherein decrease in lipid droplets in treated cells compared to untreated. Lipid droplets store cholesterol and HPβCD was able to reduce accumulation and storage of cholesterol in cancer cells.

### Staining of Lipid Rafts in Cancer Cells Using Alexa Fluor

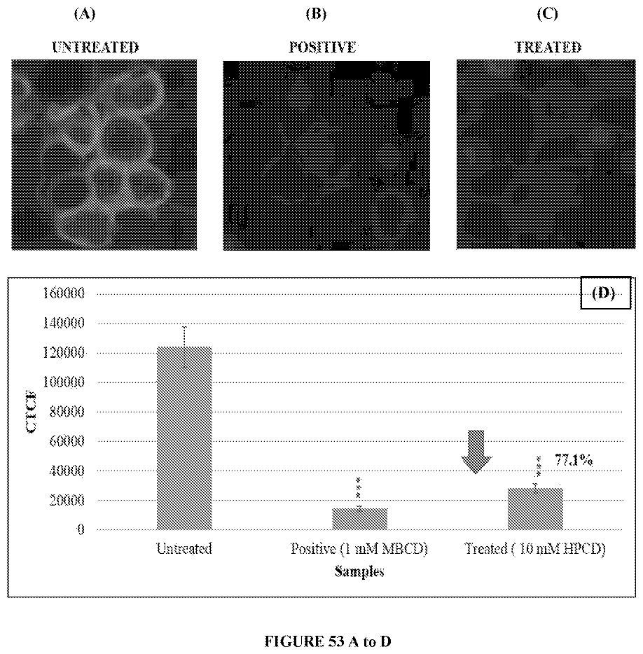

Results show decrease in lipid rafts in treated cells compared to untreated (see FIG. 53). FIG. 53 (A) to (C) shows ALEXA FLUOR staining of MCF7 cells for lipid raft dissociation, wherein (A) is untreated. (B) is positive and (C) is treated. (D) shows graphically the results wherein a decrease in lipid rafts in treated cells compared to untreated.

HPβCD is safe for use on humans and animals and provides possible solution to provide a cost effective and efficient treatment protocol for triple negative breast cancer where solid tumors typically metastasize and cause death. Moreover, since HP CD is known to be safe human clinical trials and drug registration procedures may be fast-tracked. The Applicant was surprised to unexpectedly show that HPβCD does not interact with healthy cells but has a significant impact breast cancer cells.

While the invention has been described in detail with respect to specific embodiments and/or examples thereof, it will be appreciated that those skilled in the art, upon attaining an understanding of the foregoing may readily conceive of alterations to, variations of and equivalents to these embodiments. Accordingly, the scope of the present invention should be assessed as that of the claims and any equivalents thereto, which claims are appended hereto.

1. A method comprising administering 2-hydroxypropyl-β-cyclodextrin (HPβCD) for use in the treatment of triple negative breast cancer into the body of a human or animal subject in need of the treatment.

2. The method according to claim 1, wherein the 2-hydroxypropyl-β-cyclodextrin (HPβCD) is formulated as a pharmaceutical composition.

3. The method according to claim 2, wherein the pharmaceutical composition is for administration via a parenteral and/or non-parenteral route.

4. The method according to claim 3, wherein the pharmaceutical composition is administered via parenteral administration selected from at least one of the group consisting of: intravenous, intramuscular, and implantation into the human or animal body.

5. The method according to claim 3, wherein the pharmaceutical composition is administered via non-parenteral administration selected from at least one of the group consisting of: oral-, rectal-, vaginal-, sublingual-, buccal-, and intranasal-delivery of the pharmaceutical composition into the human or animal body.

6. The method according to claim 2, wherein the pharmaceutical composition further includes an excipient and/or additional active pharmaceutical ingredients.

7. The method according to claim 3, wherein the pharmaceutical composition further includes an excipient and/or additional active pharmaceutical ingredients.

8. The method according to claim 4, wherein the pharmaceutical composition further includes an excipient and/or additional active pharmaceutical ingredients.

9. The method according to claim 5, wherein the pharmaceutical composition further includes an excipient and/or additional active pharmaceutical ingredients.
